(19) United States
(12) Patent Application Publication
(10) Pub. No.: US 2026/0290548 A1
(43) Pub. Date: Sep. 24, 2026

(54) SYSTEM

(71) Applicant: SoftBank Group Corp., Tokyo (JP)

(72) Inventor: Keisuke SAKATA, Tokyo (JP)

(73) Assignee: SoftBank Group Corp., Tokyo (JP)

(21) Appl. No.: 19/568,810

(22) Filed: Mar. 17, 2026

(30) Foreign Application Priority Data
Mar. 19, 2025 (JP) 2025-045160

(51) Int. Cl. G16H 20/60; G06F 40/40; G09B 5/06; G16H 20/30

(52) U.S. Cl. CPC G16H 20/60; G09B 5/06; G16H 20/30; G06F 40/40

(57) ABSTRACT

A system includes a processor that is configured to datafy, for a user, at least a hobby, a health status, a favorite food, a sport, and a place, datafy past life contents of the user, acquire additional information from an external information source, analyze collected data to predict a behavioral pattern of the user, integrate an analysis result to generate a user profile, create a prompt sentence for input to a generative AI model, acquire weather information, present an optimal life plan based on the hobby, the health status, the favorite food, the sport, the place, the past life contents, the additional information, the user profile, and the weather information, and present an output result from the generative AI model to the user.

### CROSS-REFERENCE TO RELATED APPLICATION

This application is based on and claims priority under 35 USC 119 from Japanese Patent Application No. 2025-045160 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional lifestyle support systems generally focus on only a subset of user-related information, such as basic preferences or simple health indicators, and provide limited recommendations that are not fully personalized. Such systems typically do not comprehensively integrate heterogeneous data sources including detailed user preferences, past life contents, external information sources, and weather information, nor do they systematically predict user behavioral patterns. Consequently, existing systems often fail to generate life plans that simultaneously reflect the user's hobbies, health status, favorite foods, sports, places, and historical behavior in a coherent manner. Furthermore, these systems do not effectively utilize generative AI models through well-structured prompts, and therefore cannot derive high-quality, context-aware content for lifestyle proposals. As a result, users may receive fragmented or suboptimal suggestions that do not sufficiently support expenditure control or stress relief while maintaining a lifestyle suitable for the user. There is therefore a need for a system capable of aggregating diverse user-related and environmental data, predicting user behavioral patterns, constructing appropriate prompts for generative AI models, and providing an integrated, optimized life plan including, for example, meals, shopping, and store selection, thereby improving the overall quality and personalization of lifestyle support.

### SUMMARY

To solve the above-described problems, the present invention provides a system comprising a processor, wherein the processor is configured to datafy, for a user, at least a hobby, a health status, a favorite food, a sport, and a place, and to datafy past life contents of the user. The processor is further configured to acquire additional information from an external information source, such as service usage records, online content, or other third-party databases, and to analyze the collected data to predict a behavioral pattern of the user. The processor integrates an analysis result of the collected and analyzed data to generate a user profile representing a comprehensive and structured model of the user's lifestyle and tendencies. Based on the generated user profile and the additional information, the processor creates a prompt sentence suitable for input to a generative AI model, thereby enabling the generative AI model to output contextually appropriate lifestyle support content. Moreover, the processor acquires weather information corresponding at least to a target period for the life plan, and presents an optimal life plan based on the hobby, the health status, the favorite food, the sport, the place, the past life contents, the additional information, the user profile, and the weather information. The processor further presents an output result from the generative AI model to the user. In one embodiment, the processor is configured to include, in the life plan, at least one of a meal, shopping, and selection of a store, and in another embodiment, the processor is configured to suppress expenditure while enabling stress relief for the user in a manner consistent with a life suitable for the user. Through these configurations, the system can generate and provide highly personalized and balanced lifestyle proposals that utilize both deterministic data analysis and generative AI capabilities.

The term “processor” refers to any hardware and/or software processing unit, including but not limited to a CPU, GPU, microcontroller, server computer, or a combination thereof, that executes instructions to perform the operations described in the present specification.

The term “datafy” refers to converting information, such as a user's hobby, health status, favorite food, sport, place, or past life contents, into a structured data format that can be stored, processed, and analyzed by the system.

The term “hobby” refers to an activity or interest that a user voluntarily engages in for enjoyment or personal fulfillment, such as running, reading, cooking, or similar leisure activities.

The term “health status” refers to information representing a user's physical or mental condition, including but not limited to qualitative or quantitative indicators such as “good,” “normal,” “poor,” medical conditions, or wellness metrics.

The term “favorite food” refers to a type or category of food that a user particularly prefers or frequently chooses, such as sushi, pasta, or other specific dishes or cuisines.

The term “sport” refers to any form of physical exercise or athletic activity in which a user participates or has an interest, including but not limited to running, swimming, tennis, or team sports.

The term “place” refers to a geographic location associated with a user, including but not limited to home, workplace, parks, restaurants, stores, or other locations that the user visits or prefers.

The term “past life contents” refers to historical records of a user's lifestyle and behaviors, including but not limited to consumption history, activity history, movement history, and usage history of services or facilities.

The term “external information source” refers to any system, service, database, API, or sensor outside the core system that provides additional information related to the user or environment, such as credit card systems, online services, map services, or other third-party data providers.

The term “additional information” refers to information acquired from an external information source that complements or extends user-related data, such as transaction history, online behavior, facility usage, or content viewing records.

The term “collected data” refers to all data obtained and stored by the system, including user profile data, past life contents, additional information from external information sources, and weather information.

The term “behavioral pattern” refers to a tendency or regularity in a user's actions or choices identified by analyzing collected data, such as typical times and places of activities, spending habits, or preferred modes of stress relief.

The term “user profile” refers to a structured representation of a user generated by integrating analysis results of collected data, including at least the user's preferences, tendencies, behavioral patterns, and lifestyle characteristics.

The term “prompt sentence” refers to a text or structured input string that is generated by the processor and supplied to a generative AI model to elicit an output that is tailored to the user profile and contextual information.

The term “generative AI model” refers to an artificial intelligence model, such as a large language model or other generative model, that generates text, recommendations, or other content in response to a prompt sentence.

The term “weather information” refers to data indicating meteorological conditions relevant to a user's life plan, including but not limited to weather forecasts, temperature, precipitation, and other climate-related parameters for a target time and place.

The term “life plan” refers to a set of proposed activities, schedules, or recommendations generated by the processor for a user, based on collected data and analysis, including but not limited to proposals regarding meals, shopping, store selection, leisure activities, and rest.

The term “meal” refers to an eating-related component of a life plan, including recommendations of specific food types, menus, restaurants, or timing of eating.

The term “shopping” refers to an activity component of a life plan in which a user purchases goods or services, including recommendations for what to buy, when to buy, and where to buy.

The term “selection of a store” refers to the process or result of choosing one or more physical or online stores to be recommended to a user for meals, shopping, or services within the life plan.

The term “expenditure” refers to monetary amounts spent or to be spent by a user, including expenses for meals, shopping, services, entertainment, and other lifestyle-related payments.

The term “stress relief” refers to activities or conditions that help a user reduce psychological or physical stress, such as leisure activities, relaxation methods, exercise, or entertainment recommended by the system.

The term “life suitable for the user” refers to a lifestyle pattern or set of activities that matches the user's preferences, health status, behavioral patterns, and constraints, while maintaining or improving the user's well-being.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

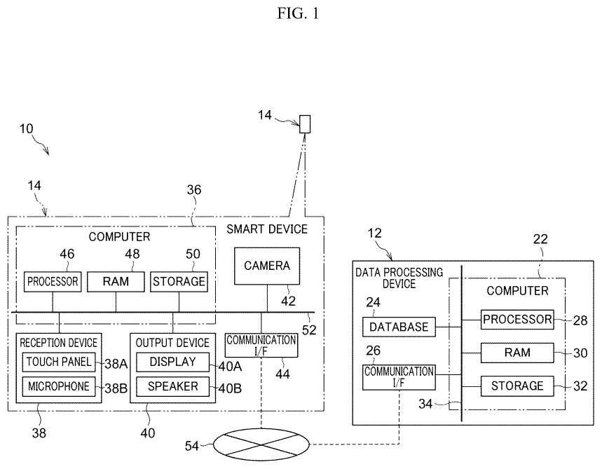

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

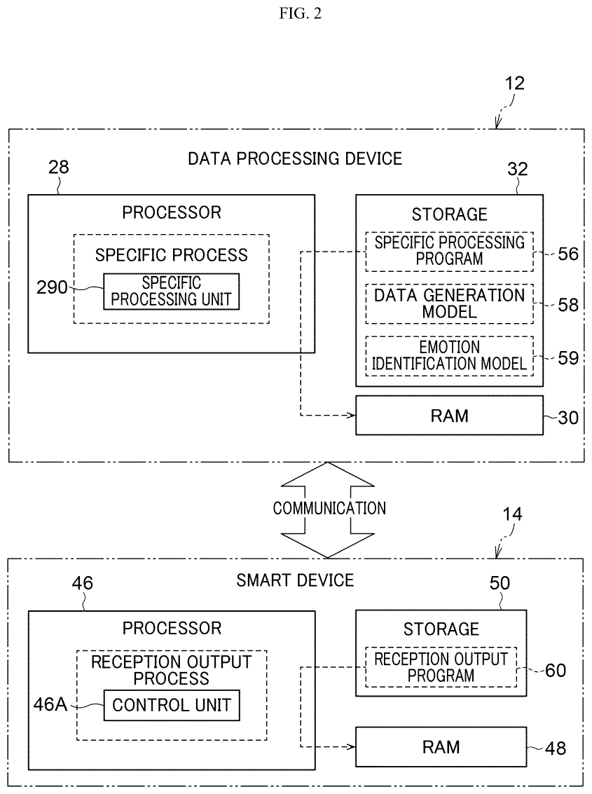

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented lifestyle recommendation systems typically rely on a limited subset of user input data, such as coarse user profile fields and static preference settings, and generate recommendations using fixed rule sets or simple scoring mechanisms. Such systems suffer from several technical problems in terms of data handling and processing. First, the systems do not efficiently integrate heterogeneous data sources, including user-provided information, historical behavioral data, and externally acquired environment information such as location context and weather conditions. As a result, the systems process fragmented datasets in separate pipelines, which increases memory usage, degrades cache locality, and leads to redundant data transformations.

Second, the systems lack a structured mechanism to transform high-dimensional behavioral and contextual data into machine-interpretable feature information and further into natural-language input suitable for a generative AI model. In many implementations, static and manually crafted prompts are used for generative models, ignoring the underlying user-specific behavior patterns and context. This creates a technical inefficiency in that a large-scale generative AI model is invoked with under-specified input, producing verbose but misaligned output, which then requires extensive post-processing on the server side. Such post-processing increases processor load, causes unnecessary network traffic between the server and the client device, and increases latency experienced by the user.

Third, existing systems generally do not couple predictive analytics (such as machine learning-based behavior pattern estimation) with automatic prompt sentence construction and structured life plan generation. Instead, predictive components and generative components operate as separate subsystems with minimal information sharing. As a consequence, the server must manage multiple inconsistent internal representations of the user state, which leads to overhead in transforming and synchronizing data structures, and complicates scaling and optimization of the system.

Fourth, conventional systems do not provide a technical mechanism for systematically incorporating constraint conditions, such as expenditure constraints and stress-reduction requirements, into an end-to-end pipeline that spans feature extraction, profile generation, prompt generation, and life-plan construction. Constraint handling is often performed as an ad-hoc filtering layer at the end of the pipeline, which discards parts of the generative output and re-invokes the model. This results in repeated computation, inefficient utilization of the generative AI model, and inconsistent behavior from the perspective of the user.

Accordingly, there is a need for a computer-implemented technique that, in a unified and structured manner, (i) acquires and integrates multi-type user data and external information into tabular data, (ii) generates feature information and evaluation information using a machine learning model, (iii) constructs user profile information in a storage-efficient representation, (iv) automatically generates a prompt sentence tailored to the user profile for a generative AI model, and (v) generates and refines a time-structured life plan while satisfying constraint conditions. Such a technique should improve the efficiency, scalability, and responsiveness of the underlying information processing system, reduce redundant processing and network usage, and provide more accurate and context-aware results with reduced computational overhead.

The specific processing by the specific processing unit 290 of the data processing device 12 in Example 1 is realized by the following means.

The present invention provides a server comprising a processor and a storage device, the processor being configured to receive, from a terminal operated by a user, information regarding user preferences including hobby information, health condition information, preference information, physical activity information, and location preference information, and to convert the received information into electronic information; to obtain, from the terminal or from a data source, history information regarding past life activities of the user, and to convert the history information into electronic information; to acquire, via a communication network from an external information source, additional information including position information, environment information, and facility information, the additional information being acquired based on position information and behavior history of the user; to integrate the electronic information, the history information, and the additional information into tabular data stored in a memory structure, and to generate feature information from the tabular data, the feature information indicating at least behavior frequency, visit type, time-zone characteristic, and preference tendency of the user; to input the feature information into a machine learning model stored in the storage device, to estimate behavior patterns and preference tendencies of the user, and to calculate evaluation information including at least a health orientation level, an activity orientation level, and a preference intensity level; to combine the evaluation information with the information received from the user to generate profile information including attribute information and behavior characteristic information of the user, and to store the profile information in the storage device in association with a user identifier; to read the profile information from the storage device and generate description information that describes, in a natural language, the attribute information and the behavior characteristic information of the user; to insert the description information into a predetermined instruction template stored in the storage device to automatically generate a prompt sentence for input to a generative AI model; to acquire weather information from a weather information source via the communication network and associate the weather information with the profile information; to generate, based on the profile information and the weather information, a life plan including at least meal content information, physical activity information, leisure activity information, and candidate visit location information, the life plan being structured by time zone and represented in a machine-processable format; to transmit the prompt sentence to the generative AI model through an interface module, and to obtain, from the generative AI model, response information including natural language text that complements or details content of the life plan; and to integrate the life plan with the response information to generate presentation information for the user, and to transmit the presentation information to the terminal so that the presentation information is presented to the user. This enables the server to implement an integrated data processing pipeline that (i) unifies heterogeneous user and environment data into a compact tabular representation, (ii) automatically derives user-specific feature information and evaluation information via a machine learning model, (iii) constructs a storage-efficient user profile, (iv) generates a context-rich prompt sentence that efficiently conditions a generative AI model, and (v) produces a constraint-aware, time-structured life plan with reduced redundant computation, improved use of processing resources and communication bandwidth, and enhanced responsiveness and accuracy of the computer-implemented lifestyle recommendation.

The term “processor” refers to a hardware computation unit or a set of hardware computation units, such as a central processing unit, a graphics processing unit, or another programmable processing circuitry, capable of executing instructions to perform arithmetic operations, logical operations, control operations, and data transfer operations.

The term “storage device” refers to a hardware data storage resource, such as a volatile memory, a non-volatile memory, a magnetic storage device, or a semiconductor storage device, that stores instructions, parameters, models, and data structures used by the processor.

The term “terminal” refers to an information processing apparatus operated by a user, such as a client computer, a mobile communication device, or a tablet device, that communicates with a server via a communication network.

The term “user” refers to a human individual who operates the terminal and provides information such as preferences, behavior history, and constraints, and to whom a life plan or recommendation is presented.

The term “user preferences” refers to information representing likes, dislikes, interests, or tendencies of the user, including but not limited to hobby information, health condition information, preference information, physical activity information, and location preference information.

The term “hobby information” refers to data indicating activities or interests voluntarily pursued by the user for enjoyment or leisure, such as sports, arts, or outdoor activities.

The term “health condition information” refers to data representing the physical or mental health state of the user, including self-reported health status, medical-related lifestyle information, or activity levels related to health.

The term “preference information” refers to data indicating the user's favored items, categories, or styles, such as preferred foods, preferred types of services, preferred activity styles, or preferred environments.

The term “physical activity information” refers to data representing movement-related behavior of the user, such as exercise frequency, type of exercise, duration of exercise, or daily activity patterns.

The term “location preference information” refers to data representing places or types of places favored or frequently visited by the user, such as preferred neighborhoods, facility categories, or environmental conditions.

The term “history information” refers to data representing past life activities of the user, including temporal sequences of actions, visited places, used services, and other behavior records acquired from the terminal or other data sources.

The term “electronic information” refers to information converted into a digital representation that can be processed, stored, or transmitted by an information processing apparatus.

The term “external information source” refers to a computing resource, database, or service that is external to the server and that provides additional data such as position information, environment information, or facility information via a communication network.

The term “position information” refers to data indicating geographic or spatial coordinates, location identifiers, or area identifiers related to the position of the user or a facility.

The term “environment information” refers to data describing conditions surrounding a position or a time, including weather conditions, temperature, congestion levels, or other contextual attributes of an environment.

The term “facility information” refers to data representing places that provide goods or services, such as stores, restaurants, exercise facilities, leisure facilities, or public facilities, including their categories, attributes, and operating conditions.

The term “behavior history” refers to a chronological record of actions or movements of the user, such as visit events, usage events, or travel paths, associated with time and place information.

The term “tabular data” refers to a structured data representation in which records are arranged in rows and attributes are represented as columns, enabling the processor to efficiently perform operations such as sorting, filtering, aggregation, and feature extraction.

The term “feature information” refers to derived data obtained from raw or integrated data that quantitatively or qualitatively represents characteristics of the user's behavior, preferences, or context, and that is suitable as input to a machine learning model.

The term “behavior frequency” refers to a numerical indication of how often a specific behavior or activity of the user occurs within a given time period.

The term “visit type” refers to a classification attribute indicative of a category or type of a visited location or facility, such as a leisure facility, a commercial facility, or a health-related facility.

The term “time-zone characteristic” refers to information representing temporal patterns of the user's behavior, such as activity distribution across different times of day or days of the week.

The term “preference tendency” refers to a quantified or categorized inclination of the user toward specific items, activities, or environments, inferred from data such as frequency, duration, or explicit ratings.

The term “machine learning model” refers to a computational model that has been trained using training data to learn parameters for mapping input feature information to output values, such as classifications, scores, or predicted attributes.

The term “behavior patterns” refers to recurring or characteristic sequences or combinations of actions, visits, or activities performed by the user, as inferred by analysis or a machine learning model.

The term “evaluation information” refers to data generated by the machine learning model that represents assessed levels, scores, or categories of certain user attributes, such as health orientation level, activity orientation level, or preference intensity level.

The term “health orientation level” refers to a value or category that indicates a degree to which the user's behavior and preferences are oriented toward maintaining or improving health.

The term “activity orientation level” refers to a value or category that indicates a degree to which the user prefers or engages in active behaviors, such as exercise or outdoor activities.

The term “preference intensity level” refers to a value or category that indicates the strength of the user's liking or inclination toward a particular item, service, or activity.

The term “profile information” refers to structured data that summarizes attributes and behavior characteristic information of the user, including evaluation information, preferences, and other relevant properties, and that is stored in association with a user identifier.

The term “attribute information” refers to data representing relatively stable characteristics of the user, such as demographic attributes, general preferences, or long-term tendencies.

The term “behavior characteristic information” refers to data representing features of the user's actions, habits, or patterns, such as frequencies, durations, and typical contexts of activities.

The term “user identifier” refers to data that uniquely identifies the user within the system, such as a numeric identifier, an alphanumeric code, or another unique token.

The term “description information” refers to natural language text generated by the processor that describes, in human-readable form, the attribute information and the behavior characteristic information of the user.

The term “instruction template” refers to a predefined natural language pattern or skeleton that includes one or more insertion positions for variable elements and that is used to construct a complete prompt sentence for a generative AI model.

The term “prompt sentence” refers to a natural language instruction generated by the processor and supplied as input to a generative AI model to condition, guide, or constrain the output generated by the generative AI model.

The term “generative AI model” refers to a computational model, typically implemented as a neural network, that generates natural language text or other content in response to input instructions, context, or conditions.

The term “weather information” refers to data describing atmospheric conditions for a particular place and time, including attributes such as temperature, precipitation, wind, humidity, and general weather state.

The term “life plan” refers to structured information describing recommended future actions or schedules for the user, including at least meal content information, physical activity information, leisure activity information, and candidate visit location information, arranged by time.

The term “meal content information” refers to data describing recommended eating or drinking items, menus, or nutritional compositions for particular times or situations.

The term “physical activity information” refers to data describing recommended exercise or movement activities, including type, intensity, timing, and duration.

The term “leisure activity information” refers to data describing recommended non-work and non-obligatory activities aimed at relaxation, enjoyment, or hobbies, including suggested types and timing of such activities.

The term “candidate visit location information” refers to data indicating one or more locations or categories of locations that are recommended for the user to visit, such as suggested areas, facility categories, or specific facilities.

The term “response information” refers to data received from the generative AI model in response to the prompt sentence, including natural language text that complements or details content of the life plan.

The term “presentation information” refers to data generated by the processor by integrating the life plan and the response information, formatted so that it can be displayed or otherwise presented to the user by the terminal.

The term “constraint conditions regarding expenditure amount” refers to one or more limitations or target values related to expected or allowable spending within a certain period or for certain activities.

The term “conditions regarding stress reduction” refers to requirements or objectives relating to reducing psychological or physical stress experienced by the user, such as increasing relaxing activities or balancing workloads.

The term “communication network” refers to any wired or wireless data communication infrastructure that enables data exchange between the server, the terminal, and external information sources.

In one embodiment, a server, a terminal, and a communication network cooperate to implement the claimed system. The server includes at least one processor, a main memory, a non-volatile storage device, and a network interface. The terminal includes at least one processor, a memory, an input interface, a display device, and a communication interface. The terminal and the server are connected via a wired or wireless communication network. The server executes an operating system and middleware and runs application software that implements a user data acquisition module, a data integration module, a feature extraction module, a machine learning inference module, a profile management module, a prompt generation module, a generative AI interface module, and a presentation generation module. The server uses a database management system to store profile information, feature information, and history information in a persistent manner.

The terminal executes a client application implemented, for example, as a web application or a native application. The client application includes a user interface module that presents input forms and recommendation results to the user, and a communication module that exchanges structured messages with the server over the communication network.

The user operates the terminal to enter user preferences. The terminal displays input fields for hobby information, health condition information, preference information such as favorite foods, physical activity information such as exercise habits, and location preference information such as preferred types of places. The terminal converts the entered values into digital form and stores them temporarily in the memory of the terminal. The terminal then transmits the digital information to the server through the communication network using a structured communication protocol.

The server receives the user preferences and converts the received data into internal data structures, such as records and tabular data held in the main memory. The server further obtains history information regarding past life activities of the user from the terminal or from other data sources. The server may, for example, obtain sequences of visited locations and timestamps collected by the terminal using a positioning mechanism, and obtain usage records of facilities or services. The server converts the history information into digital form and merges it with the user preferences.

The server acquires additional information from an external information source. The external information source may comprise a map information service, an environment information service, and a facility information service that provide position information, environment information, and facility information via the communication network. The server sends requests including a user identifier and location context to the external information source and receives response data describing, for example, categories of visited locations, operating hours of facilities, and environmental conditions at corresponding times and places. The server converts the response data into tabular data, associates it with the user identifier, and integrates it with the user preferences and the history information.

The server represents the integrated data as tabular data in a main-memory structure, for example, as a table in which each row corresponds to a behavior event and each column corresponds to an attribute, such as event timestamp, facility category, stay duration, expenditure amount, and environmental context. The server uses this tabular representation to perform aggregation and transformation operations. For example, the server aggregates rows by facility category and time interval to compute behavior frequency values, such as a number of gym visits per week or a number of restaurant visits per month. The server also computes time-zone characteristics by counting or averaging activities in predefined time ranges, such as morning, daytime, evening, and night. The server further quantifies preference tendencies by calculating relative frequencies and durations associated with particular categories of foods, activities, or locations.

The server generates feature information from the tabular data. The feature information is represented as a feature vector that includes numerical values and encoded categorical values. For example, the feature vector may include elements that represent gym visit frequency per week, average exercise duration per session, ratio of healthy food selections, number of outdoor activities per week, and indicators for preferred facility categories. The server may encode categorical attributes using one-hot encoding or ordinal encoding to obtain a fixed-length numeric vector that can be input to a machine learning model. This specific construction of feature information improves processing efficiency and cache utilization because it reduces disparate data structures to a compact, contiguous representation suitable for vectorized arithmetic operations on the processor.

The server uses a machine learning model to estimate behavior patterns and preference tendencies of the user from the feature information. In one embodiment, the server stores a trained model in the storage device. The model may be implemented as a neural network, such as a multilayer perceptron with an input layer that receives the feature vector, one or more hidden layers using rectified linear unit activation functions, and an output layer that outputs continuous scores and categorical probabilities. Alternatively, the model may be implemented as an ensemble model, such as a gradient-boosted decision tree model, that maps the feature vector to predicted scores.

The server loads the model parameters from the storage device into the main memory and performs inference by supplying the feature vector to the model. During training of the model, the server has used historical labeled data to compute a loss function, such as a mean squared error for regression targets or a cross-entropy loss for classification targets. The server has executed an optimization algorithm, such as stochastic gradient descent with momentum or an adaptive moment estimation method, to update model weights. In some embodiments, the server has employed data augmentation techniques, such as random perturbation of time intervals or aggregation windows, to improve robustness of the model to noise in behavior records. As a result, the trained model can accurately map the feature information to evaluation information.

During inference, the server computes, from the model outputs, evaluation information including a health orientation level, an activity orientation level, and a preference intensity level. The server may, for example, output a health orientation level as a real value between 0 and 1, and classify that value into categorical levels such as “low,” “medium,” or “high.” The server may compute the activity orientation level from features describing outdoor and indoor activities, and compute the preference intensity level from ratios of specific preferences, such as preference for particular foods or facility types.

The server combines the evaluation information with the user preferences and the history information to generate profile information. The server defines a data structure in which each user is associated with a profile record, and the profile record includes attribute information such as demographic attributes, long-term preferences, and stable habits, and behavior characteristic information such as typical time-zone characteristics, frequently visited category types, and computed orientation levels. The server stores the profile information in the storage device, for example, in a relational database table indexed by a user identifier. The storage of profile information in this compact form reduces both main-memory footprint and persistent-storage footprint, improving overall data management efficiency.

The server reads the profile information from the storage device when a recommendation is requested. The server generates description information that describes, in a natural language, the attribute information and the behavior characteristic information of the user. For example, the server may generate a description such as: “The user is highly health-oriented, performs cycling several times per week, prefers light meals and sushi, and typically finishes work at 18:00 on weekdays.” The server produces the description by applying a rule-based formatting procedure that maps numerical scores and categorical values in the profile information to predefined textual expressions. This rule-based mapping is non-trivial and differs from mere human summarization because the mapping is optimized for compressing high-dimensional feature vectors into controlled natural language text of bounded length, thereby improving communication efficiency with downstream generative AI models.

The server uses an instruction template to construct a prompt sentence for a generative AI model. The server stores, in the storage device, one or more instruction templates that include insertion positions for variable content. For example, a template may be:
“You are a professional lifestyle planner. Based on the following user profile, create a detailed one-week life plan that optimizes health, enjoyment, and practicality.

User profile: [PROFILE_TEXT]

Please specify daily meal ideas, exercise recommendations, and relaxing evening activities, including approximate times and example locations or types of places. Output the plan in clear, structured text.”

The server inserts the description information into the insertion position [PROFILE_TEXT] to form a complete prompt sentence. Another example of a prompt sentence generated by the server is:
“You are a nutrition-focused chef. Please suggest five healthy dinner recipes for a user who is highly health-conscious, enjoys cycling, and likes sushi.

### Requirements:

Avoid deep-fried dishes.
Limit heavy cream and butter.
Use ingredients that are easy to find in a typical supermarket in Japan.

For each recipe, output a title, a brief description, a list of main ingredients, and simple cooking instructions.”

The server thereby generates a prompt sentence that is specific to the user's profile and that constrains the expected output format and content. This automatic construction of prompt sentences from profile information is performed according to deterministic rules and template structures, and it improves the effectiveness of the generative AI model by reducing ambiguity in the input space. From a computer-technical perspective, this leads to more predictable output distributions from the generative model and reduces the number of iterations and network round-trips required to obtain usable results, thereby lowering communication load and processing time.

The server acquires weather information from a weather information source via the communication network and associates the weather information with the profile information. For example, the server may obtain forecast data for the user's region for each day and time slot in the planning horizon. The server merges weather conditions, such as expected rain, temperature, and wind, into the profile context so that subsequent life plan generation can avoid weather-incompatible suggestions. This integration of environmental context into the digital representation reduces the need for manual adjustment and additional filtering, thereby improving overall correctness and stability of the generated plans.

The server generates a life plan based on the profile information and the weather information. The server represents the life plan in a machine-processable structure that associates time-zone slots with recommended actions. A record for each time slot may include fields for meal content information, physical activity information, leisure activity information, and candidate visit location information. The server may generate an initial life plan using algorithmic rules that map evaluation information and constraints to action allocations. For example, when the health orientation level and activity orientation level are high, and the weather is clear in the evening, the server may assign an outdoor physical activity such as cycling to evening slots and assign light meals with balanced nutrition to meal slots. When the weather indicates rain, the server may assign indoor activities and alternative meal choices consistent with the user's preferences. This algorithmic allocation is designed to maintain a bounded complexity by using pre-computed mappings and threshold rules, so that the processor can generate an initial plan with low latency.

The server optionally considers constraint conditions regarding expenditure amount and conditions regarding stress reduction when generating or modifying the life plan. The server may include, in the profile information, a target budget per week and indicators of stress levels. The server then uses these values to limit the selection of candidate visit location information and meal content information, preferring low-cost options or free activities while maintaining sufficient leisure and relaxation content. By embedding the constraints at the time of plan generation rather than applying them only as post-filters, the server avoids generating large amounts of unusable plan data, which improves computational efficiency and reduces wasteful use of processing cycles.

The server uses the generative AI model as a specialized component to refine and detail the life plan. The server transmits the prompt sentence to the generative AI model and receives response information including natural language text. The generative AI model may be implemented as a transformer-based neural network that has been pre-trained on large corpora of text and fine-tuned for instruction following. The server accesses the model via an application programming interface exposed by a model hosting platform. The server specifies model parameters such as maximum output length, sampling temperature, and top-k or top-p sampling parameters to control output diversity and determinism. Because the server supplies structured and constrained prompt sentences, the generative AI model can generate detailed text that is aligned with the initial life plan, thereby minimizing the need for large-scale post-editing by the server.

The server receives the response information and integrates the generated natural language text with the machine-processable life plan representation. The server may, for example, parse the generated text using recognition rules that identify day names and time references and map them to the internal time-slot structure. The server thereby associates descriptive sentences and recipe details with specific plan elements, such as breakfast, lunch, and dinner slots. By maintaining this mapping between text and structured data, the server can later perform efficient updates or adapt the plan when new constraints or weather changes arise, without regenerating all textual content.

The server generates presentation information for the user by combining the structured life plan and the integrated natural language text into a format suitable for display at the terminal. The server may represent the presentation information in a hierarchical structure, such as days containing time slots, each time slot containing meal details, physical activity descriptions, and leisure activity descriptions. The server transmits the presentation information to the terminal via the communication network.

The terminal receives the presentation information and displays it to the user. The terminal may display, for each day, textual descriptions and lists such as “Recommended recipe: avocado salad with grilled salmon; main ingredients: salmon, avocado, lettuce, olive oil,” “Exercise: 30-minute cycling in the evening three times per week,” and “Weekend plan: cycling along a riverside route and dinner at a sushi restaurant.” The terminal may additionally show icons or maps based on candidate visit location information. By offloading the heavy machine learning inference and data integration processing to the server and using the terminal primarily as a presentation and input device, the system optimizes distribution of computational load and reduces resource requirements on the terminal side.

The server, the terminal, and the generative AI model together achieve technical effects that go beyond mere automation of human planning. The server's conversion of heterogeneous logs and external data into tabular feature information enables efficient vectorized processing and improves cache locality, which in turn increases processing throughput and reduces energy consumption per recommendation. The integration of machine learning-based evaluation information and rule-based prompt construction produces higher-quality and more context-consistent generative outputs, thereby reducing the number of interactions with the generative AI model and lowering communication bandwidth usage. The use of constraint conditions at the profile level ensures that the generative AI model is conditioned with precise constraints in the prompt sentence, reducing the need for repeated correction and truncation of generated text.

In another embodiment, the server may employ a different model architecture, such as a recurrent neural network or a convolutional neural network, for generating evaluation information, or may use a different external information source, such as a transportation information service, while still applying the same overall framework of feature extraction, profile generation, prompt sentence construction, and life plan generation. In another embodiment, the terminal may perform part of the prompt generation or display processing, or may provide interactive controls for the user to adjust evaluation thresholds or constraint conditions, and the server may adapt its processing according to the updated parameters.

These alternative configurations remain within the scope of the inventive concept, because they preserve the core technical structure of integrating multi-source data, generating machine-interpretable feature information, creating user-specific profile information, automatically constructing a prompt sentence for a generative AI model, and generating a time-structured life plan with improved computational efficiency and accuracy.

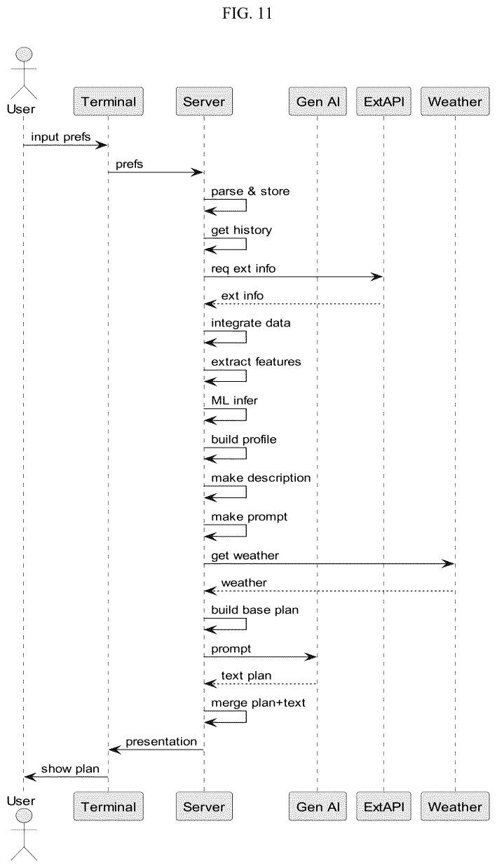

The following describes the processing flow using FIG. 11.

### Step 1:

The user operates the terminal to input initial preference information. The terminal displays input fields for hobby information, health condition information, preference information, physical activity information, and location preference information. The input is user-entered text or selections (input), such as “hobby: cycling,” “health: good,” and “favorite food: sushi.” The terminal converts the received input into structured electronic data, such as key-value pairs, and stores the data in a local memory buffer (output). The terminal thereby transforms human-readable input into machine-processable preference data.

### Step 2:

The terminal transmits the structured preference data to the server. The input to this step is the structured preference data and a user identifier stored in the terminal. The terminal encapsulates this data into a request message, for example an HTTP request body formatted in a structured format, and sends the message through a communication interface to the server (output). The terminal may add metadata such as timestamps and device identifiers. The data processing consists of serializing the structured data, attaching headers, and performing network transmission.

### Step 3:

The server receives the preference data from the terminal. The input to this step is the request message containing the serialized preference data. The server parses the message, validates required fields, and converts the serialized data back into an internal data structure, such as a record object or an in-memory table (output). The server performs data checking, type conversion, and normalization, for example by trimming strings and converting categories to standardized codes.

### Step 4:

The server acquires history information related to the user. The input to this step is the user identifier included in the parsed preference data. The server queries internal logs or an external data source to obtain past behavior records, such as visited locations, activity timestamps, and facility usage. The server receives raw log entries or event records (output) and converts them into a consistent digital format by extracting relevant fields and discarding noise. The data processing includes filtering by user identifier and time range, and mapping service-specific fields into a unified schema.

### Step 5:

The server acquires additional information from an external information source. The input to this step is the user's position context or behavior history obtained in the previous step. The server constructs one or more request messages to external services, specifying, for example, coordinates and time intervals, and sends them via the communication network. The server receives response data such as environmental conditions, facility attributes, and regional context (output). The server parses the responses, converts them into tabular entries, and associates each entry with the user identifier and the corresponding event. This data processing comprises parsing, type conversion, and joining external attributes to internal event records.

### Step 6:

The server integrates the preference data, history information, and additional information into tabular data. The input to this step is the normalized preference record, the unified history records, and the external information entries. The server constructs a table in memory where each row represents an event or aggregated unit, and columns include attributes such as event time, facility category, environmental attributes, and preference codes. The output is a consolidated tabular dataset keyed by user identifier. The server performs join operations, grouping by keys, and filling missing values with default codes or interpolated values.

### Step 7:

The server generates feature information from the integrated tabular data. The input to this step is the consolidated table for a given user. The server executes aggregation operations such as counting events per category per time interval, averaging durations, and computing ratios of specific behaviors. The server transforms categorical attributes into numerical encodings, and arranges the resulting values into a fixed-length feature vector (output). The data processing includes computing behavior frequency metrics, time-zone statistics, preference ratios, and encoding them as numerical elements suitable for model input.

### Step 8:

The server applies a machine learning model to the feature information. The input to this step is the feature vector associated with the user. The server loads model parameters from storage into memory and performs a forward pass through the model, which may be a neural network or another predictive model. The server calculates intermediate activations and final output values, such as predicted scores and probabilities (output). The data operations include matrix multiplications, non-linear activation function evaluations, and normalization of output scores.

### Step 9:

The server computes evaluation information from the model output. The input to this step is the raw outputs of the machine learning model, such as continuous scores and classification probabilities. The server converts these outputs into interpretable metrics, for example by mapping score ranges to discrete categories like “high” or “low,” and by combining related outputs into composite indices. The output is evaluation information including a health orientation level, an activity orientation level, and a preference intensity level. The data processing includes thresholding, scaling, and aggregating model outputs into labeled evaluation fields.

### Step 10:

The server generates profile information for the user. The input to this step is the evaluation information and the user's original preference data and behavior history summary. The server constructs a profile record that includes attribute information such as stable preferences and demographic attributes, and behavior characteristic information such as dominant facility categories and time-zone usage patterns (output). The server stores this profile record in the storage device associated with the user identifier. The data processing includes field selection, structuring into a defined schema, and writing the structured record to persistent storage.

### Step 11:

The server prepares description information in natural language. The input to this step is the stored profile information retrieved from the storage device. The server maps numerical and categorical fields to textual expressions using predefined rules, for example transforming a high health orientation level into the phrase “highly health-conscious.” The server concatenates these expressions according to a formatting pattern to form a coherent descriptive text (output). The data processing includes look-ups in mapping tables, conditional text selection based on thresholds, and string concatenation.

### Step 12:

The server constructs a prompt sentence for a generative AI model. The input to this step is the description information and a predefined instruction template stored in the server. The server inserts the description information into the placeholder region of the template, and appends additional instructions specifying the required output format and constraints. The output is a complete prompt sentence. For example, the server generates a prompt such as:
“You are a professional lifestyle planner. Based on the following user profile, create a detailed one-week life plan that optimizes health, enjoyment, and practicality.

User profile: highly health-conscious; enjoys cycling several times per week; likes sushi; typically finishes work at 18:00 on weekdays.

Please specify daily meal ideas, exercise recommendations, and relaxing evening activities, including approximate times and example locations or types of places. Output the plan in clear, structured text.”

The data processing consists of template selection, placeholder substitution, and string assembly.

### Step 13:

The server acquires weather information and merges it with the profile context. The input to this step is a request period and the user's location information from the profile. The server sends queries to a weather information source and receives forecast data (output) for each day and time slot. The server associates the forecast entries with planning time slots by matching timestamps and regions. The data processing includes parsing weather responses, indexing by time and place, and augmenting the planning context with weather attributes such as precipitation probability and temperature ranges.

### Step 14:

The server generates an initial life plan in structured form. The input to this step is the profile information, the evaluation information, and the merged weather context. The server applies rule-based allocation logic that maps orientation levels and constraints to activity categories for each time slot, taking into account weather conditions and budget constraints. The server outputs a machine-processable structure in which each time slot is assigned meal content information, physical activity information, leisure activity information, and candidate visit location information. The data processing includes selecting actions from predefined sets, applying priority rules, and storing the selected actions in a time-indexed data structure.

### Step 15:

The server sends the prompt sentence to the generative AI model and obtains response information. The input to this step is the constructed prompt sentence and, optionally, a summary of the initial life plan. The server formats a request for the generative AI model, including the prompt text and control parameters such as maximum output length and sampling settings, and transmits the request via a model interface. The server receives a response containing generated natural language text (output), which details or refines plan elements such as daily schedules and recipes. The data processing includes request packaging, network communication, and extraction of the generated text from the model's response format.

### Step 16:

The server integrates the generated text with the structured life plan. The input to this step is the initial life plan structure and the natural language text returned by the generative AI model. The server parses the text using pattern recognition rules that detect day names, time expressions, and semantic segments such as “breakfast,” “exercise,” and “dinner.” The server links each text segment to corresponding time slots and action categories within the structured life plan and updates or annotates the plan fields with the detailed descriptions (output). The data processing includes text segmentation, keyword matching, time normalization, and updating of structured records.

### Step 17:

The server generates presentation information for the terminal. The input to this step is the updated structured life plan with associated natural language descriptions. The server arranges the plan into a hierarchical representation suitable for display, for example grouping by day and by time slot, and attaches display metadata such as section titles and ordering keys. The output is a presentation data object that contains all text and structural information required by the terminal. The data processing includes hierarchical structuring, sorting by time, and formatting fields for user-interface components.

### Step 18:

The server transmits the presentation information to the terminal. The input to this step is the presentation data object constructed by the server. The server serializes the object into a transmission format and sends it by a response message over the communication network. The output is a delivered response message containing the structured plan and all associated information. The data processing includes serialization, header attachment, and network transmission.

### Step 19:

The terminal receives and displays the presentation information. The input to this step is the response message carrying the serialized presentation data. The terminal parses the message, reconstructs the hierarchical structure, and maps each part to corresponding user interface elements. The output is a visual display of the life plan on the terminal's screen, including text such as “Recommended recipe: avocado salad with grilled salmon; main ingredients: salmon, avocado, lettuce, olive oil” and “Exercise: 30-minute cycling in the evening three times per week.” The data processing includes deserialization, UI layout generation, and rendering of text and optional icons.

### Step 20:

The user reviews the displayed plan and provides optional feedback. The input to this step is the on-screen presentation of the plan. The user may select items, indicate preferences, or request changes via interaction controls. The terminal converts the user's interactions into digital feedback data (output), such as ratings or acceptance flags. The data processing includes capturing input events, updating local state, and, if necessary, preparing additional messages for transmission to the server for subsequent refinement of the profile information and future plan generation.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional lifestyle recommendation systems typically rely on static rule sets or simple scoring mechanisms that are applied independently to heterogeneous user data such as preferences, health status, purchase history, and environmental conditions. These systems generally treat user profile information, historical behavior information, and external context information as loosely coupled data sources and do not convert them into a unified, machine-learning-ready representation. As a result, they cannot effectively capture complex, non-linear relationships between a user's long-term behavioral tendencies, dietary constraints, and contextual factors such as weather or time, and they often produce generic, low-precision recommendations.

Furthermore, known systems that employ machine learning models for recommendation are usually optimized for a single task, such as predicting a product purchase or recommending a single meal. They do not generate a consistent, multi-domain lifestyle plan that integrates meal planning, purchasing activities, and facility visits under explicit constraints such as budget limitations and health conditions. In addition, such systems typically lack a feedback loop that structurally incorporates user selection information and evaluation information into both the data representation and the learning process, which limits their ability to improve recommendation accuracy over time.

In recent developments, generative AI models have been used to produce natural language explanations or high-level suggestions. However, integration of such models with structured, machine-learned lifestyle planning remains ad hoc. Existing approaches usually send raw or loosely summarized user information to a generative model, without generating a well-formed prompt sentence that systematically encodes the outputs of a deterministic learned model, external environmental information, and user attributes. This leads to unstable or inconsistent quality in generated explanations and proposals, and it does not improve the underlying computational behavior of the recommendation engine itself.

Moreover, there is a technical need to efficiently manage and transform large volumes of heterogeneous user-related data—such as structured profile attributes, transaction logs, and interaction feedback—into numerical feature quantities suitable for machine learning processing, while maintaining the ability to update models with new feedback in an online or periodic manner. Conventional architectures do not tightly couple the data storage management, feature generation, model execution, and generative AI prompting processes into a coherent computation pipeline. Consequently, they suffer from redundancy in data transformations, increased latency, and difficulty in maintaining consistency between the structured plan used for computation and the narrative output presented to the user.

Accordingly, there is a need for an improved computer-implemented system that: (i) acquires and stores heterogeneous user-related information as structured and history data in an integrated information storage device; (ii) converts such data into feature quantities for machine learning; (iii) executes a learned model to generate candidate item sequences and lifestyle plan information under explicit constraints; (iv) automatically constructs a prompt sentence for a generative AI model based on the machine-learned lifestyle plan and user attributes; (v) incorporates environmental information such as weather conditions into both the planning and prompting stages; and (vi) closes the loop by using user selection and evaluation information to update the stored history data and improve subsequent feature generation and model behavior. By addressing these shortcomings, the present invention improves the functioning of computer-based lifestyle and meal planning systems, leading to more accurate, context-aware, and adaptively improving recommendations.

The specific processing by the specific processing unit 290 of the data processing device 12 in Application Example 1 is realized by the following means.

The present invention provides a server comprising a processor and a non-transitory storage device storing instructions which, when executed by the processor, cause the processor to acquire preference information, health information, food-related information, physical-activity-related information, and movement-related information regarding a user and record the information as structured data in the storage device; record past behavior information and transaction information regarding the user as history data in the storage device; manage an information storage region that stores the structured data and the history data; extract the structured data and the history data from the information storage region and convert the extracted data into numerical data sequences as feature quantities for machine learning processing; execute a learned model including a machine learning algorithm with the feature quantities as input to estimate a behavior tendency and a dietary tendency of the user and to generate a candidate item sequence to which a suitability degree is assigned for the user; apply constraint conditions and operation rules to the candidate item sequence and generate lifestyle plan information including meal plan information by selecting candidates based on a health condition and a preference condition of the user; generate a prompt sentence to be used as input to a generative information processing model based on the lifestyle plan information and attribute information regarding the user; input the prompt sentence and the lifestyle plan information into an external generative information processing model and acquire a generation result including explanation information and additional proposal information corresponding to the lifestyle plan information; acquire weather information from an environmental information source and reflect the weather information in generation of the lifestyle plan information and in creation of the prompt sentence; transmit the lifestyle plan information and the generation result to a user information terminal and cause the user information terminal to present the lifestyle plan information and the generation result visually; and acquire selection information and evaluation information regarding the lifestyle plan information from the user information terminal, record the selection information and the evaluation information as part of the history data in the storage device, and reflect the selection information and the evaluation information in learning of the learned model or in generation of the feature quantities. This enables an integrated computational pipeline in which heterogeneous user-related data are transformed into machine-learning-ready feature quantities, used to compute constrained lifestyle plans, and then encoded into structured prompt sentences for a generative AI model, while user feedback and environmental information are cyclically incorporated, thereby improving the accuracy, adaptability, and computational efficiency of computer-implemented lifestyle and meal planning.

The term “user” refers to an individual or an organization for which lifestyle plan information is generated and to which recommendations and explanations are presented.

The term “preference information” refers to information indicating likes, dislikes, priorities, or tendencies of a user with respect to items such as foods, activities, locations, or services.

The term “health information” refers to information indicating a physical or mental condition of a user, including at least one of medical conditions, dietary restrictions, biometric indicators, or wellness goals.

The term “food-related information” refers to information associated with intake of food or beverages by a user, including at least one of preferred dishes, ingredients, nutritional constraints, and historical meal selections.

The term “physical-activity-related information” refers to information indicating a user's activities that consume physical energy, including at least one of exercise habits, sport activities, daily step counts, and activity intensity levels.

The term “movement-related information” refers to information indicating spatial or temporal movement of a user, including at least one of visited locations, travel routes, commute patterns, and time-of-visit records.

The term “structured data” refers to data that are organized according to a predefined schema or format, such as records, fields, and data types, enabling deterministic access, processing, and storage by a computer system.

The term “past behavior information” refers to information indicating historical actions or activities of a user, including at least one of consumption actions, visits to facilities, and usage of services.

The term “transaction information” refers to information indicating economic or commercial interactions by a user, including at least one of purchase records, payment amounts, item identifiers, and transaction timestamps.

The term “history data” refers to data in which past behavior information and transaction information of a user are recorded and stored in association with the user over time.

The term “information storage device” refers to a hardware and software combination that provides non-transitory storage of data, including at least one of memory devices, magnetic storage devices, semiconductor storage devices, and database management systems.

The term “information storage region” refers to a logical or physical portion of an information storage device in which structured data and history data are stored and managed for subsequent retrieval and processing.

The term “feature quantities” refers to numerical data values derived from structured data and history data, which are formatted as input variables for machine learning processing by a learned model.

The term “numerical data sequences” refers to ordered sets of numeric values that represent encoded or transformed versions of original user-related information for use in computational algorithms.

The term “machine learning processing” refers to computational processing in which a model adjusts or applies parameters to map input feature quantities to predicted outputs according to patterns learned from example data.

The term “learned model” refers to a model that has been trained using a machine learning algorithm and example data to represent relationships between input feature quantities and output variables such as recommendations or scores.

The term “machine learning algorithm” refers to an algorithm that optimizes parameters of a model based on example data, including at least one of regression algorithms, classification algorithms, clustering algorithms, and neural network algorithms.

The term “behavior tendency” refers to a pattern or likelihood of future actions of a user inferred from feature quantities, including at least one of preferred activity types, typical time-of-day behaviors, and facility-visit patterns.

The term “dietary tendency” refers to a pattern or likelihood of food intake behavior of a user, including at least one of preferred cuisines, common nutrient ranges, and frequently selected dishes.

The term “candidate item sequence” refers to an ordered list of items that may be recommended to a user, including at least one of food items, activities, products, or facility visits.

The term “suitability degree” refers to a value or score indicating a relative appropriateness or preference level of a candidate item for a specific user under given conditions.

The term “constraint conditions” refers to conditions that restrict or limit selection of candidate items, including at least one of health-related constraints, budget constraints, schedule constraints, or environmental constraints.

The term “operation rules” refers to predefined rules for controlling generation or filtering of candidate items, including at least one of business rules, policy rules, or application-specific rules.

The term “lifestyle plan information” refers to structured information representing a planned set of actions or choices for a user over a period, including at least one of meal plan information, purchase plan information, and facility-visit candidate information.

The term “meal plan information” refers to information defining one or more recommended meals for a user, including at least one of dish identifiers, meal times, nutritional values, and portion sizes.

The term “health condition” refers to a state or requirement derived from health information of a user, including at least one of dietary restrictions, maximum intake thresholds, or medically related constraints.

The term “preference condition” refers to a condition derived from preference information of a user, including at least one of required inclusion of certain categories, exclusion of disliked items, or prioritization of favored attributes.

The term “attribute information” refers to information that characterizes a user, including at least one of demographic attributes, lifestyle attributes, and stable preference attributes.

The term “prompt sentence” refers to a text string constructed according to a predetermined format and including at least a summary of lifestyle plan information and attribute information, which is supplied as input to a generative information processing model.

The term “generative information processing model” refers to a model that generates text or other content based on an input sequence, including at least one of language models, sequence-to-sequence models, and generative neural network models.

The term “generation result” refers to output information produced by a generative information processing model in response to a prompt sentence, including at least one of explanation information and additional proposal information.

The term “explanation information” refers to natural language information that explains or justifies elements of lifestyle plan information for presentation to a user.

The term “additional proposal information” refers to supplementary recommendations or alternatives generated by a generative information processing model in relation to lifestyle plan information.

The term “environmental information source” refers to an external data source that provides information regarding external conditions, including at least one of weather data sources, location data sources, and schedule data sources.

The term “weather information” refers to information indicating meteorological conditions at a time and place relevant to the user, including at least one of temperature, precipitation, humidity, and weather forecasts.

The term “user information terminal” refers to an electronic apparatus operated by or accessible to a user, including at least one of a smartphone, tablet device, personal computer, or other communication-enabled terminal.

The term “selection information” refers to information indicating which elements of lifestyle plan information are accepted, rejected, or modified by a user via a user information terminal.

The term “evaluation information” refers to feedback information from a user regarding satisfaction, preference level, or suitability of lifestyle plan information, including at least one of rating values, textual comments, and reaction indicators.

The term “visual presentation” refers to display of information in a human-readable form on a screen of a user information terminal, including at least one of text, icons, lists, charts, and graphical user interface elements.

In one embodiment, a server includes a processor, a main memory, a non-transitory storage device, a network interface, and an operating system. The server executes application software implemented, for example, using a general-purpose programming language and a web application framework to provide an application programming interface (API). The server further cooperates with a database management system to store user-related information, and with a machine learning runtime library to execute a learned model. The server communicates with one or more terminals operated by a user via a communication network.

A terminal includes at least one processor, a memory, a display, an input interface, and a communication interface. The terminal executes client software, such as a native application or a web application, that presents a graphical user interface (GUI) to the user. The terminal allows the user to input preference information, health information, food-related information, physical-activity-related information, and movement-related information, and to view lifestyle plan information and explanation information received from the server.

A user operates the terminal to register profile attributes, specify dietary preferences and constraints, and provide feedback on presented lifestyle plans. The user may input structured selections via form elements in the GUI and may additionally input free text such as “I am currently on a low-carb diet and I prefer Japanese food.” The user may subsequently provide feedback text such as “Please avoid spicy foods” or “Increase vegetable-based dishes at dinner.”

The server stores structured data and history data in a relational database executed by the database management system. The server defines tables for user profiles, health profiles, preferences, transaction records, visit records, and feedback records. The server associates each record with a user identifier and timestamps. The server represents preference information, health information, and other profile attributes in normalized relational form, using foreign keys to link user records to preference categories, health condition categories, and location categories.

The server converts the structured data and history data into feature quantities suitable for machine learning processing. The server defines a data schema in which user-related information is mapped to fixed-length feature vectors. The server encodes categorical attributes (such as cuisine types, health condition categories, or activity categories) using methods such as one-hot encoding or embedding indices. The server normalizes numerical attributes (such as daily calorie intake, expenditure amounts, and number of facility visits) using scaling functions (for example, min-max scaling or z-score normalization). The server constructs numerical data sequences by concatenating feature vectors corresponding to recent time windows of user behavior, such as behavior in the past N days or past M transactions. The server executes a learned model implemented as a neural network architecture, for example a multi-layer feedforward neural network or a sequence model such as a recurrent neural network or a transformer-based network. The server defines an input layer that receives the feature quantities, one or more hidden layers that perform linear transformations and non-linear activation functions (such as rectified linear unit, sigmoid, or hyperbolic tangent functions), and an output layer that produces scores for candidate items. The server may additionally implement attention mechanisms to weight different components of the feature quantities, such as distinguishing the influence of recent behaviors from older behaviors.

The server trains the learned model offline or periodically using training datasets stored in the database. The server uses a loss function such as cross-entropy loss or mean squared error, depending on the prediction target. The server updates the weights of the neural network using gradient-based optimization methods, such as stochastic gradient descent, Adam, or RMSprop. The server computes gradients of the loss function with respect to the model parameters by backpropagation. The server may perform data augmentation in the feature space, for example by sampling different time windows, shuffling sequence segments, or artificially perturbing numeric values within realistic ranges to improve robustness. The server uses training labels derived from historical user interactions. For example, the server treats items that were selected or highly rated by the user as positive examples and items that were ignored or negatively rated as negative examples. The server uses these labels to adjust the suitability degree predicted for candidate items. The server optionally segments users into cohorts based on stable attributes, and trains cohort-specific or shared models to increase accuracy.

The server generates a candidate item sequence by applying the trained model to the feature quantities of a given user. The server obtains raw scores from the output layer, interprets them as relevance or suitability degrees, and sorts candidate items by descending score. The server then applies constraint conditions and operation rules that are stored in configuration tables or rule engines. For example, the server rejects items exceeding a maximum carbohydrate threshold derived from the user's health information, rejects items containing ingredients flagged as disliked or allergenic in the preference information, and enforces budget constraints by filtering out combinations of items whose total price exceeds a specified daily or weekly expenditure limit.

The server generates lifestyle plan information by grouping and selecting candidate items in accordance with time slots, categories, and constraints. For meal plan information, the server assigns specific dishes to breakfast, lunch, and dinner for one or more days. The server computes aggregated nutritional values such as total daily calories, total carbohydrates, and macro-nutrient breakdown, and verifies that these fall within ranges derived from the user's health condition. The server also generates purchase plan information by mapping recommended dishes to required ingredients and identifying potential purchase items. The server further generates facility-visit candidate information, such as recommended restaurants or stores, by correlating candidate items with facility records in the database.

The server constructs a prompt sentence for a generative AI model based on the lifestyle plan information and attribute information of the user. The server formats the prompt sentence as natural language text that encodes structured constraints, context, and plan content. For example, the server may generate a prompt sentence such as:
“Based on the following user profile and data, generate a 3-day meal plan with breakfast, lunch, and dinner for each day. The user has a low-carb preference and a goal of maintaining stable blood sugar levels. Prior order history shows frequent selection of grilled fish, tofu, and vegetable-based dishes. Please provide dish names, short descriptions, and approximate calorie and carbohydrate amounts for each meal.”

Alternatively, the server may generate a shorter prompt sentence such as:
“Using the user's health status and past order history, propose an optimal meal plan for the next two days. The user is currently on a carbohydrate-restricted diet and prefers Japanese cuisine. Include at least two alternative options for each meal.”

The server conditions the text generation by explicitly inserting machine-derived metrics, such as numeric carbohydrate limits, aggregate calories, or predicted satisfaction scores, into the prompt sentence. This use of machine-computed internal state as explicit text tokens improves the generative AI model's ability to respect constraints and maintain internal consistency.

The server transmits the prompt sentence and, optionally, part of the structured lifestyle plan information to an external generative AI model via an API. The generative AI model executes a sequence-to-sequence generation algorithm, typically implemented as a large-scale neural network such as a transformer trained on large corpora. The server receives the generation result comprising explanation information and additional proposal information.

The explanation information may describe, in human-readable language, why particular dishes or activities were chosen, how the plan satisfies health constraints, and how the plan respects user preferences. The additional proposal information may contain alternative dishes, variations, or tips to adjust the plan.

The server performs post-processing on the generation result to ensure consistency and compliance with stored constraints. The server may parse numerical values from the generated text and compare them with computed totals, truncating or correcting inconsistent descriptions. The server may also remove or flag any additional proposals that violate the stored health condition or budget constraints.

The server retrieves weather information from an environmental information source, such as an online weather service, using a network call. The server parses the weather information to obtain attributes such as predicted temperature, precipitation probability, and weather codes.

The server includes these attributes as part of the feature quantities or as additional contextual conditions. For example, during hot weather, the server may adjust the candidate item sequence by increasing the suitability degrees for lighter meals or indoor activities. The server also uses the weather information when constructing the prompt sentence that is sent to the generative AI model, by including phrases such as “The weather will be hot and humid, so please suggest refreshing, light meals and mostly indoor activities.”

The terminal receives the lifestyle plan information and generation result from the server via the communication interface. The terminal parses the structured data and renders the content on the display using the GUI. The terminal displays lists or calendar views of meals, graphical indicators of nutritional content, and textual explanation information from the generative AI model. The terminal allows the user to interact with each proposed item through selection buttons, rating controls, and comment input fields.

The user interacts with the displayed lifestyle plan information by accepting certain proposals, rejecting others, requesting replacements, or adjusting constraints such as maximum daily expense or calorie limits. The user inputs these interactions via the GUI using touch gestures, mouse clicks, or keyboard input. The user may, for example, tap a “Replace dinner” button, adjust a slider representing maximum daily cost, or enter a comment such as “Prefer non-fried dishes for dinner.”

The terminal aggregates the user interactions as selection information and evaluation information and transmits them to the server. The server stores these as feedback records in the database and updates aggregated metrics such as per-item acceptance rates, user-specific affinity scores for ingredients, and satisfaction scores. The server uses this updated history data in subsequent feature generation and model inference, so that the learned model gradually adjusts to the user's evolving preferences.

This configuration improves computer technology in several respects. The server implements a tightly integrated data pipeline that converts heterogeneous user-related information into machine-learning-ready feature quantities in a consistent and repeatable manner. By fixing the feature schema and defining explicit mappings from database fields to neural network inputs, the server reduces data conversion overhead and improves cache locality, resulting in faster inference. The use of normalized vectors and batched inference enables the server to perform matrix operations efficiently using hardware acceleration, which reduces processing latency.

The server improves accuracy by jointly modeling multiple dimensions of user behavior and context, including preferences, health constraints, transaction history, movement patterns, and weather conditions, instead of treating them as independent inputs. The learned model captures complex interactions between these dimensions, which a rule-based system or manual human processing cannot efficiently compute. The system further improves data management by storing feature-relevant aggregates and intermediate representations, enabling incremental updates to feature quantities without scanning the entire history data.

The server reduces communication load by sending only compact structured results and condensed explanation text to the terminal, rather than raw historical data. The server performs heavy computation, including feature generation, model inference, and prompt sentence construction, on the server side, which allows the terminal to be implemented on resource-constrained devices with limited processing capability. This division of labor enhances overall system throughput and responsiveness.

The use of a generative AI model is not merely an automation of human explanation, but an architectural component that is closely integrated with the deterministic learned model. The server uses internal predictions, constraints, and aggregated statistics as explicit tokens in the prompt sentence, creating a non-traditional coupling between structured recommendation computation and natural language generation. This coupling leads to more consistent and constraint-aware narrative outputs, which directly support the usability of the system without requiring additional human curation.

The server differs from conventional human-based or rule-based processes by applying non-linear optimization and learned weightings that adapt dynamically to new feedback. The server uses explicit objective functions and gradient-based training, which systematically reduce prediction error and refine the suitability degrees assigned to items. The server also uses non-standard rules that combine learned scores with explicit constraint checks and contextual conditions (such as weather), which yield lifestyle plans that are both feasible and personalized.

In alternative embodiments, the server may implement different neural network architectures, such as convolutional neural networks for processing spatially structured movement patterns, or graph neural networks to model relationships between users, items, and facilities. The server may use different loss functions, such as ranking-based loss for ordered candidate item sequences, or multi-task loss that combines prediction of satisfaction scores and adherence to health constraints. The server may support online learning, in which model parameters are updated incrementally upon receiving new feedback, or federated learning, in which partial updates are computed on terminals and aggregated on the server to improve privacy.

In another embodiment, the server may store precomputed embeddings for items, ingredients, and facilities, and may update these embeddings using techniques such as matrix factorization or deep metric learning. The server may also vary the construction of prompt sentences depending on user language preferences, information density preferences, or accessibility requirements, while maintaining the core structure that encodes the machine-computed plan and constraints.

Through these implementations and variations, the server, the terminal, and the user cooperate to realize a lifestyle planning system in which complex heterogeneous data are transformed, analyzed, and presented in a technically improved manner. The integrated feature generation, learned model inference, generative AI prompting, and feedback incorporation collectively provide improved computational efficiency, higher recommendation precision, and adaptive behavior that cannot be achieved by conventional manual or rule-based approaches.

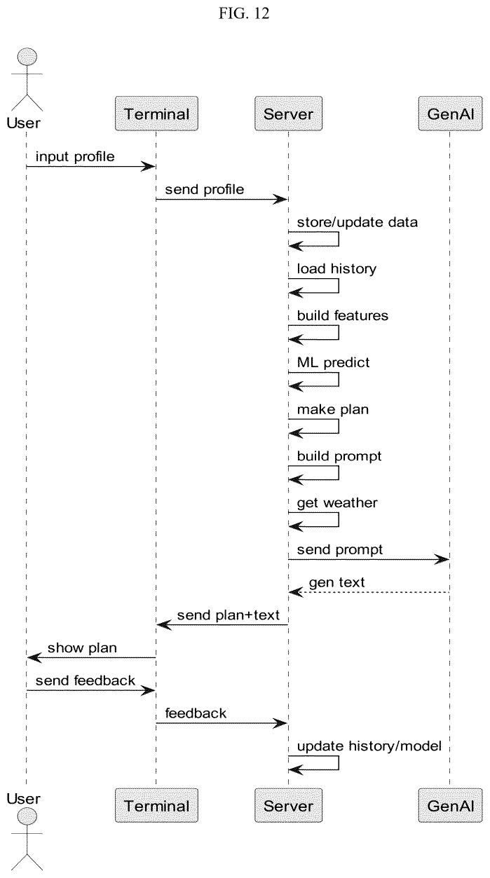

The following describes the processing flow using FIG. 12.

### Step 1:

The user operates the terminal and inputs profile information including preference information, health information, food-related information, physical-activity-related information, and movement-related information through graphical user interface components such as text fields, check boxes, sliders, and location selectors. As input, the terminal receives raw user entries (strings, numeric values, selections, and GPS-derived locations). The terminal converts these heterogeneous inputs into a structured internal representation, such as key-value pairs and typed objects, and validates formats (for example, checking that age is numeric and that dietary constraints are selected from predefined categories). As output, the terminal generates a structured data object representing the user profile.

### Step 2:

The terminal packages the structured data object as a request payload and attaches metadata such as user identifier, device identifier, timestamp, and locale. As input, the terminal uses the structured profile representation produced in Step 1. The terminal serializes this representation into a machine-readable message format and establishes a secure network connection to the server using a communication interface. As output, the terminal transmits a request containing the serialized user profile data to a designated API endpoint on the server.

### Step 3:

The server receives the request via a network interface and parses the serialized payload using input-handling routines of the application framework. As input, the server obtains the serialized profile data and metadata from the terminal. The server deserializes the payload into internal data structures, performs validation (for example, checking mandatory fields and allowable ranges), and standardizes units (such as converting calories and distances into canonical units). As output, the server produces normalized structured data suitable for storage and subsequent processing.

### Step 4:

The server stores the normalized structured data in an information storage device managed by a database management system. As input, the server uses the validated profile attributes and associated metadata. The server executes database operations to insert or update records in user profile tables, health profile tables, preference tables, and movement or activity tables, linking them with a user identifier. The server may also update index structures to accelerate subsequent queries. As output, the server maintains persistently stored structured data that reflect the current state of the user profile.

### Step 5:

The server retrieves history data from the information storage device to prepare for feature generation. As input, the server uses the user identifier and query parameters such as time windows or maximum record counts. The server issues database queries to obtain past behavior information, transaction records, facility-visit records, and previous feedback records. The server optionally computes aggregates, such as total expenditure over a recent period, average daily calories, frequency of specific cuisines, and acceptance rates for recommended items. As output, the server produces a collection of structured history data and aggregate statistics associated with the user.

### Step 6:

The server generates feature quantities for machine learning processing from the structured data and history data. As input, the server uses the current profile information from Step 4 and the history data and aggregates from Step 5. The server encodes categorical attributes (such as cuisine categories, health condition categories, and activity types) into numerical vectors, normalizes numerical attributes (such as calorie values, expenditure amounts, and counts), and assembles them into fixed-length feature vectors. The server may also construct temporal sequences by stacking vectors representing multiple past time intervals. As output, the server obtains numerical data sequences formatted as machine-learning-ready input tensors.

### Step 7:

The server executes a learned model on the generated feature quantities to calculate suitability degrees for candidate items. As input, the server provides the feature tensors from Step 6 to a neural network model comprising an input layer, multiple hidden layers with non-linear activations, and an output layer. The server performs matrix multiplications, bias additions, and activation function evaluations to propagate the feature tensors through the network. As output, the server generates raw scores or probabilities for a set of candidate items representing possible meals, activities, purchases, or facility visits.

### Step 8:

The server applies constraint conditions and operation rules to the candidate items scored by the learned model. As input, the server uses the raw scores from Step 7, the user's health condition and preference condition from the structured data, and any stored policy rules or budget limits. The server ranks items according to their scores, discards items violating constraints (such as exceeding nutrient thresholds, including disallowed ingredients, or breaching budget limits), and re-ranks or re-weights remaining items if necessary. As output, the server produces a filtered and ordered candidate item sequence in which each item is associated with an adjusted suitability degree.

### Step 9:

The server constructs lifestyle plan information, including meal plan information, from the filtered candidate item sequence. As input, the server uses the ordered candidate items from Step 8, time-slot definitions (such as breakfast, lunch, and dinner), and planning parameters (such as number of days to plan). The server assigns items to specific time slots and days, ensuring that daily nutritional totals and cost totals remain within the ranges implied by health and budget constraints. The server calculates aggregated metrics such as daily calories, daily carbohydrate totals, and total projected expenditure. As output, the server produces structured lifestyle plan information that groups candidate items into a coherent schedule and includes derived metrics.

### Step 10:

The server generates a prompt sentence for a generative AI model based on the lifestyle plan information and attribute information of the user. As input, the server uses the structured lifestyle plan from Step 9, user attribute information such as dietary goals and cuisine preferences, and relevant contextual parameters such as time horizon. The server formats these inputs into natural language text according to a predefined template, inserting explicit numerical values and constraints. For example, the server may output a prompt such as: “Based on the following user profile and data, generate a 3-day meal plan with breakfast, lunch, and dinner for each day. The user has a low-carb preference and a goal of maintaining stable blood sugar levels. Prior order history shows frequent selection of grilled fish, tofu, and vegetable-based dishes. Please provide dish names, short descriptions, and approximate calorie and carbohydrate amounts for each meal.” As output, the server obtains a textual prompt sentence that encodes machine-computed structure and constraints.

### Step 11:

The server enriches the prompt sentence and the lifestyle plan information with environmental context such as weather information. As input, the server uses the prompt sentence from Step 10 and invokes an environmental information source to retrieve weather information for relevant dates and locations. The server parses the weather data and determines conditions such as temperature ranges and precipitation probabilities. The server then modifies or supplements the prompt sentence by adding descriptions like “The weather will be hot and humid, so please suggest refreshing, light meals and mostly indoor activities.”

As output, the server produces a context-aware prompt sentence and may also adjust the lifestyle plan information to align with the environmental conditions.

### Step 12:

The server transmits the context-aware prompt sentence and associated structured information to an external generative AI model and receives a generation result. As input, the server provides the prompt sentence from Step 11 and, optionally, a summary of the lifestyle plan information as additional context. The server sends these inputs via a network call to a generative AI service that executes a language-generation algorithm. The server then receives a textual generation result that includes explanation information describing the rationale of the plan and additional proposal information suggesting alternatives or enhancements. As output, the server obtains processed natural language text aligned with the structured lifestyle plan.

### Step 13:

The server performs post-processing on the generation result to ensure compatibility with constraints and to prepare data for presentation. As input, the server uses the generation result from Step 12 and the structured lifestyle plan from Step 9. The server may parse the generated text to detect numerical assertions, compare them with stored metrics such as calories or costs, and remove or adjust inconsistent parts. The server associates segments of explanation text with specific plan elements (for example, particular meals or activities). As output, the server produces a combined representation comprising the lifestyle plan information and aligned explanation and proposal text.

### Step 14:

The server sends the lifestyle plan information and the generation result to the terminal for display. As input, the server uses the validated combined representation from Step 13 and user-specific display preferences. The server serializes the structured plan and associated texts into a response message and transmits this message through the network interface to the terminal. As output, the terminal receives a response containing all necessary information to present the plan, explanations, and alternatives.

### Step 15:

The terminal parses the received response and renders the content to the user. As input, the terminal uses the response containing structured lifestyle plan data and explanation text from Step 14. The terminal deserializes the data, maps meal and activity entries to visual components such as list items, calendar entries, and charts, and places explanation text in associated detail views or tooltips. The terminal may highlight constraints (for example, low-carb labels or budget indicators) and show aggregated metrics in dedicated panels. As output, the terminal presents an interactive visual representation of the plan and explanations on the display.

### Step 16:

The user evaluates the presented lifestyle plan and provides selection information and evaluation information via the terminal. As input, the user views the meals, activities, and recommendations displayed in Step 15. The user interacts with interface controls to accept or reject specific items, request replacements, assign ratings, and enter comments such as “Replace this dinner with a lighter option” or “Increase walking activities on weekends.” As output, the terminal records the user's choices and feedback as structured interaction events.

### Step 17:

The terminal aggregates the interaction events into a feedback payload and sends it to the server. As input, the terminal uses the collection of user actions from Step 16, including identifiers of affected plan items, ratings, and comments. The terminal encodes this information into a structured format linked to the user identifier and transmits it to a feedback-handling endpoint on the server. As output, the terminal delivers a compact set of feedback records to the server.

### Step 18:

The server updates history data and adjusts future feature generation and model behavior using the received feedback. As input, the server receives the feedback payload from Step 17. The server stores the feedback as new history records in dedicated feedback tables, updates aggregate statistics such as per-item acceptance rates and user-specific preference scores, and may mark certain constraints as strengthened or relaxed based on comments. The server incorporates these updated statistics into subsequent feature-generation steps by recalculating or augmenting feature vectors with feedback-derived values. As output, the server produces an updated state in the database and in feature computation logic, so that future executions of Step 6 and Step 7 reflect the latest user behavior and improve the accuracy and personalization of subsequent lifestyle plans.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional lifestyle recommendation systems typically rely on static rule sets or coarse user attributes, such as age group or general preferences, and therefore generate generic suggestions that are not closely aligned with an individual user's actual behavior. Such systems often treat transaction data, location data, and external environment information, including time and weather, as separate data streams, without integrating them into a unified behavioral profile. As a result, existing systems are unable to accurately model fine-grained lifestyle patterns, such as combined spending tendencies and movement tendencies over time, and cannot generate highly contextualized recommendations.

Moreover, in many existing systems, any use of a generative AI model is limited to responding to manually crafted prompts that are not systematically derived from underlying behavioral data. This results in unstable output quality, low reproducibility, and an inability to adaptively refine prompts based on updated user behavior. There is also insufficient feedback between the user's actual subsequent actions and the generation of future recommendations, causing the system to remain insensitive to behavioral changes and making it difficult to reduce computational waste and network load caused by irrelevant AI calls.

From the perspective of computer technology, there is therefore a need for improved mechanisms within an information processing apparatus to (i) automatically structure and analyze transaction history data and location data at scale, (ii) algorithmically extract lifestyle pattern information that fuses behavioral characteristics, consumption characteristics, movement patterns, and preference tendencies, (iii) dynamically generate and update prompt sentences for a generative AI model based on machine-readable profile information, and (iv) close the loop by incorporating usage result information back into storage and analysis in order to continuously update the profile and prompt generation logic. Without such mechanisms, the processing apparatus cannot efficiently allocate computational resources, cannot reliably tailor the generative AI model's output to the user's actual context, and cannot improve the technical performance of the system, including data processing efficiency, relevance of generated plans, and stability of the AI interaction workflow.

The specific processing by the specific processing unit 290 of the data processing device 12 in Example 2 is realized by the following means.

The present invention provides a server comprising a processor and a storage unit, the processor being configured to acquire and digitize usage history information and location information related to a user from a user terminal via a secure communication path; structure the usage history information and the location information and store the structured information in the storage unit by using a data management mechanism; execute an analysis program to aggregate and statistically process the stored usage history information and the stored location information to extract lifestyle pattern information including behavioral characteristics and consumption characteristics of the user; integrate the lifestyle pattern information with time information and environment information to generate profile information summarizing behavioral tendencies, movement tendencies, expenditure tendencies, and preference tendencies of the user; dynamically generate, on the basis of the profile information and the environment information, a machine-readable prompt sentence for input to a generative AI model; input the prompt sentence to the generative AI model via an interface program and acquire response information including a lifestyle plan candidate; post-process the response information to extract candidate actions, candidate facilities, and candidate time slots and to generate lifestyle plan information that conforms to the lifestyle pattern information and constraint conditions; transmit the lifestyle plan information to the user terminal for presentation to the user; and acquire usage result information representing subsequent user behavior and update at least one of the lifestyle pattern information and the generation logic of the prompt sentence based on the usage result information. This enables the server to technically improve the end-to-end lifestyle recommendation pipeline by automatically learning and maintaining a refined behavioral profile in the storage unit, by generating context-specific prompt sentences that stabilize and enhance the output of the generative AI model, by reducing unnecessary computation and network traffic through feedback-driven refinement of analysis and prompting, and by increasing the precision and relevance of lifestyle plan information delivered to the user terminal.

The term “usage history information” refers to digital information representing records of a user's past activities, including at least payment activities, purchasing activities, service usage activities, and other interactions with facilities or services, each record typically including a time, a content of the activity, and a related cost or resource consumption.

The term “location information” refers to digital information representing a geographic position associated with the user or a user terminal, including at least a coordinate, a time, and optionally an area identifier or a facility identifier derived from the coordinate.

The term “information processing apparatus” refers to an electronic data processing system including at least one processor and at least one storage unit that are configured to execute programs, store data, and perform communication with external devices.

The term “communication path” refers to a logical or physical data transmission channel between devices, including at least a network, a communication protocol, and a security layer for transferring digital information.

The term “storage unit” refers to a hardware-based memory resource, such as a semiconductor memory or a magnetic storage device, configured to store digital data and programs used by the processor.

The term “data management mechanism” refers to a logical or physical subsystem configured to manage storage, retrieval, and organization of digital data in the storage unit, including at least a data schema, indexing procedures, and access control procedures.

The term “analysis program” refers to a sequence of instructions executed by the processor for performing aggregation, statistical processing, pattern detection, and related computation on stored data.

The term “lifestyle pattern information” refers to structured digital information representing extracted regularities in a user's behavior, including at least behavioral characteristics, consumption characteristics, temporal patterns, and spatial patterns derived from usage history information and location information.

The term “behavioral characteristics” refers to attributes that describe how a user acts over time, including frequency, timing, and type of activities, such as dining, shopping, commuting, and leisure activities.

The term “consumption characteristics” refers to attributes that describe how a user spends resources, including spending amounts, spending categories, preferred price ranges, and changes in spending behavior over time.

The term “time information” refers to digital information indicating temporal aspects such as date, time of day, day of week, or period, which can be used to classify or group events.

The term “environment information” refers to digital information representing contextual conditions external to the user, including at least weather conditions, calendar information, regional events, and other environmental parameters.

The term “profile information” refers to structured digital information summarizing a user's lifestyle pattern information, including at least behavioral tendencies, movement tendencies, expenditure tendencies, and preference tendencies, in a form suitable for further processing.

The term “behavioral tendencies” refers to patterns indicating likely user behaviors under given contexts, including repeated choices of activity types, times, or locations.

The term “movement tendencies” refers to patterns indicating typical spatial transitions or stay behaviors of a user, including frequently visited areas, common routes, and usual time periods of presence in those areas.

The term “expenditure tendencies” refers to patterns indicating how a user typically allocates financial resources, including favored spending categories, typical transaction amounts, and periods of higher or lower expenditure.

The term “preference tendencies” refers to patterns indicating user likes and dislikes, including preferred facility types, service types, product categories, or environmental conditions inferred from repeated behavior.

The term “prompt sentence” refers to a sequence of natural language tokens or structured instruction tokens that is automatically generated by the processor and that is supplied as an input query to a generative AI model to cause the generative AI model to produce response information.

The term “generative AI model” refers to a machine learning model configured to generate new data, including natural language text, in response to an input, the model being trained on large-scale data and executed on a computing resource separate from or integrated with the information processing apparatus.

The term “interface program” refers to a software component executed by the processor that formats, transmits, and receives data for communication between the information processing apparatus and the generative AI model using a defined protocol.

The term “response information” refers to digital information output from the generative AI model in response to a prompt sentence, the information including at least candidate actions, candidate facilities, candidate time slots, or descriptions thereof.

The term “lifestyle plan candidate” refers to a not-yet-validated set of proposed actions, facilities, and time arrangements generated by the generative AI model based on a prompt sentence.

The term “candidate actions” refers to proposed user activities, such as visiting a facility, purchasing a product, using a service, or performing a task, that are derived from the response information.

The term “candidate facilities” refers to proposed places or service providers, such as shops, restaurants, or public facilities, that are associated with candidate actions in the response information.

The term “candidate time slots” refers to proposed temporal intervals or points in time for performing candidate actions, indicated in or derived from the response information.

The term “lifestyle plan information” refers to structured digital information generated by the processor from response information and lifestyle pattern information, the lifestyle plan information specifying at least recommended actions, recommended facilities, and recommended time slots that are adjusted to constraint conditions.

The term “constraint conditions” refers to rules or parameters restricting the generation of lifestyle plan information, including budget constraints, time constraints, distance constraints, or user-specified conditions.

The term “user terminal” refers to an endpoint device operated by a user, such as a portable computing device or a stationary computing device, that is capable of communicating with the information processing apparatus and presenting information to the user.

The term “usage result information” refers to digital information representing actual user behavior after presentation of lifestyle plan information, including at least updated usage history information and updated location information that indicate whether and how the user followed the lifestyle plan information.

The term “generation logic of the prompt sentence” refers to algorithms, rules, or parameter settings executed by the processor to construct a prompt sentence from profile information, environment information, and other context data, including selection of templates, insertion of variables, and adjustment of instruction content.

The term “secure communication path” refers to a communication path that employs at least encryption, authentication, and integrity protection to prevent unauthorized access or tampering with transmitted information between the user terminal and the information processing apparatus.

In one embodiment, a server cooperates with a terminal operated by a user to implement a lifestyle plan generation system. The server includes at least one central processing unit (CPU), a main memory, a non-volatile storage device such as a solid-state drive, and a network interface. The terminal includes at least one processor, a memory, a position sensor such as a GPS receiver, and a wireless communication interface. The server executes software components including an operating system (for example, a general-purpose server operating system), a database management system (for example, a relational database engine), a web application framework (for example, a server-side application framework), and a data analysis library (for example, a numerical computation library and a tabular data processing library). The server also interacts with a generative AI model deployed on a local accelerator device (for example, a graphics processing unit) or on a remote inference service through a network.

The terminal acquires usage history information and location information related to the user.

The terminal runs an application that calls a location service of the terminal operating system, such as a platform-specific location API, to obtain latitude, longitude, timestamp, and accuracy information at configurable intervals or when positional displacement exceeds a threshold. The terminal also acquires usage history information from a financial application or a service application by calling an application programming interface that returns transaction records including at least an activity type, an amount, a category code, a location indicator, and a timestamp. The terminal temporarily stores these records in a local structured store such as a small-scale relational database embedded in the terminal.

The terminal transmits the usage history information and the location information to the server via a secure communication path. The terminal establishes a Transport Layer Security (TLS) session with the server and serializes the data in a structured text-based format such as JavaScript Object Notation (JSON). The terminal includes a user identifier, device identifier, and authentication token in the header section of an HTTP request. The terminal sends the request to an application programming interface endpoint exposed by the server.

The server receives the request and validates the payload. The server terminates the TLS session, verifies a digital certificate, and authenticates the request using the token. The server deserializes the JSON payload into in-memory data structures in the main memory. The server executes validation routines that check the structure, types, and ranges of each field. For example, the server checks that latitude and longitude values fall within valid geographic bounds, that timestamps conform to a defined time format, and that monetary amounts are non-negative and do not exceed predefined thresholds. The server rejects invalid records and logs anomalies for diagnostic purposes.

The server structures and stores the validated data in a relational database. The server uses a data management mechanism that defines normalized table schemas such as a “transactions” table and a “location_data” table. The “transactions” table includes columns such as user_id, activity_type, merchant_category_code, amount, currency_code, timestamp, and location reference. The “location_data” table includes columns such as user_id, latitude, longitude, timestamp, and derived_cluster_id. The server executes parameterized SQL INSERT statements through a database driver, commits the transaction, and makes the data available for subsequent analytical queries.

The server retrieves data for analytical processing by executing SQL SELECT statements that filter by user identifier and time window. The server loads the result sets into a data analysis module that uses a tabular data structure similar to a DataFrame. The server converts timestamp fields into a high-resolution time representation and normalizes currency codes and amount fields. The server uses vectorized operations to group transaction records by merchant_category_code and by discrete time slots such as hourly intervals or day-of-week buckets. The server computes aggregated statistics, including count, sum, average, and variance of amounts per category and per time slot. The server also groups location records by approximate coordinates or by precomputed cluster identifiers.

The server generates lifestyle pattern information by combining statistical results from both the transaction data and the location data. The server executes a clustering algorithm such as k-means or density-based clustering on the location records. The server normalizes latitude and longitude coordinates, selects an appropriate number of clusters based on a heuristic such as the elbow method or a silhouette score, and then assigns each location record to a cluster identifier. The server calculates visit frequency, average dwell time, and temporal distribution for each cluster. The server labels representative clusters as candidate “home,” “work,” or “frequent leisure area” based on temporal patterns, such as night-time dominance for home or weekday daytime dominance for work.

The server then merges the clustered location data with the transaction data by joining on timestamp or on a location reference field. The server identifies co-occurrence patterns where certain spending categories are associated with specific clusters and time ranges. For example, the server detects that the user performs restaurant-related transactions near a particular cluster during weekday lunch intervals, or that the user frequently visits cafes within a distance threshold from a home cluster during weekend afternoons. The server encodes these patterns into lifestyle pattern information, represented as machine-readable structures containing fields for frequent_areas, preferred_time_slots, preferred_price_ranges, spending_hotspots, and mobility_patterns.

The server converts the lifestyle pattern information into profile information. The server uses rule-based logic and machine-learned thresholds to transform raw statistical values into discrete behavioral tendencies, movement tendencies, expenditure tendencies, and preference tendencies. For example, the server categorizes a spending category as a “primary preference” if its normalized frequency and total amount exceed learned thresholds. The server categorizes a geographic cluster as a “frequent area” if visits occur above a visit frequency threshold for several consecutive periods. The server stores the resulting profile information in a dedicated table or document store to allow low-latency retrieval for future processing.

The server dynamically generates a prompt sentence to be used as input to a generative AI model. The server executes a prompt construction module that uses the profile information and current environment information, such as date, time, and weather conditions, to select a template from a template library. Each template is a parameterized natural language instruction pattern. The server fills the template with concrete values derived from the profile information, such as place names, time ranges, price levels, and behavioral objectives. The server may generate, for example, a prompt sentence in the form:
“The user usually has lunch near the central business area between 11:30 and 13:30 on weekdays and prefers moderately priced casual restaurants. Based on this pattern, please propose three optimal lunch spots for this week. Include each restaurant's name, type of cuisine, approximate price range, and a short explanation of why it matches the user's preferences.”

In another case, the server may generate a prompt sentence such as:
“The user spends most weekend afternoons at home in a residential district and enjoys visiting new cafes within a 2-kilometer radius. Please suggest three new cafes the user has not visited yet, suitable for a relaxing weekend afternoon. For each cafe, provide the name, location, typical price range, and a short reason why it is recommended.”

In a further example, the server may generate a prompt sentence such as:
“The user wants to reduce monthly dining-out expenses by 20% while maintaining low stress. Based on the following summarized life pattern and spending data: frequent restaurant visits near the office on weekdays and frequent cafe visits near home on weekends, please propose a weekly life plan for meals, including when to cook at home and when to dine out, with brief justifications for each recommendation.”

The server communicates with a generative AI model using an interface program. In one embodiment, the generative AI model is a transformer-based neural network that has an encoder-decoder or decoder-only architecture with multiple attention layers, feed-forward layers, and layer normalization. The model is characterized by parameters such as the number of layers, the number of attention heads per layer, the hidden dimension size, and an activation function such as a rectified linear unit or a smooth variant. The model is trained on large corpora of natural language data using a self-supervised objective such as next-token prediction or masked-token prediction.

The server feeds the prompt sentence to the generative AI model through an application programming interface. The server sends the prompt sentence and inference parameters such as maximum output length, temperature, and top-k or top-p sampling settings. The generative AI model processes the tokenized prompt sentence by applying multi-head self-attention operations to compute contextual embeddings for each token, passing these through stacked layers to generate a probability distribution over possible next tokens. The model then selects output tokens iteratively according to the sampling strategy and constructs a response text.

The server receives the response text and executes a post-processing module. The server parses the response text and splits it into separate recommendation items by detecting enumerations or delimiter patterns. The server applies regular expression matching or simple semantic tagging to identify key fields in each recommendation, such as facility name, category, approximate price range, and explanation. If a recommendation contains a place name, the server calls an external geocoding service or an internal place database to attempt to resolve the place name to a geographic coordinate or facility identifier. The server creates lifestyle plan information by structuring the parsed recommendations into object records with fields that can be easily presented to the user or further processed.

The server transmits the lifestyle plan information to the terminal. The server either pushes a notification via a push messaging service or waits for the terminal to poll a dedicated endpoint. The terminal requests the lifestyle plan information and receives the structured recommendations from the server. The terminal displays the information in an application interface. The terminal may show a list of recommended actions, such as visiting specific restaurants or cafes, including names, categories, expected price ranges, and descriptive reasons. The terminal may additionally display map views using a map software development kit to show the routes from the current location to the recommended facilities.

The user reviews the lifestyle plan information on the terminal and optionally selects some recommendations to follow. The user may trigger additional actions such as navigation, reservation, or exclusion of certain recommendations. The user's subsequent behavior, including actual visits and spending, is again captured by the terminal as new usage history information and location information, which are sent back to the server. The server incorporates this usage result information into the database. The server re-runs its analysis routines periodically or when sufficient new data are available, updates the lifestyle pattern information and the profile information, and adjusts the parameterization of the prompt construction logic. For example, the server may lower the prominence of facilities that the user repeatedly ignores or increase the weight of categories that the user consistently follows.

In one embodiment, the server improves computational efficiency and recommendation precision by using specific data structures and algorithmic optimizations. The server stores time-series data in partitioned tables indexed by user_id and timestamp to accelerate range queries. The server precomputes features such as per-category spending rates and cluster-level dwell times and stores them in feature tables to avoid repeated recalculation. The server implements a caching mechanism for frequently used profile information and template selections so that subsequent prompt generation requests for similar contexts can be served quickly without performing a full re-analysis. By doing so, the server reduces the number of expensive database queries and reduces network calls to the generative AI model, which decreases overall latency and network traffic.

The server also improves accuracy and stability of generative AI model outputs by employing a feedback mechanism that monitors outcomes of prior recommendations. The server logs which recommendations were followed by the user based on subsequent location data and usage history information. The server correlates these outcomes with features of the corresponding prompt sentences and generative AI responses. The server adjusts parameters of the prompt construction logic, such as the inclusion or exclusion of certain details, the explicitness of constraints, or the requested output format, so that future prompt sentences align more closely with recommendation patterns that have historically produced positive outcomes. This non-traditional prompt optimization based on closed-loop behavioral feedback enables the system to refine how it controls the generative AI model, thereby improving the technical behavior of the model from the perspective of the server.

In a further embodiment, the server executes a model selection or weighting algorithm that chooses among different generative AI models or different decoding parameter sets according to the complexity of the requested lifestyle plan and the available computational resources.

The server may maintain metadata describing past performance of different model configurations in terms of user acceptance rates and computational cost. The server uses this metadata as features in a decision function that selects a model configuration for each new request. This enables the system to balance precision and resource usage in a dynamic way that would not be feasible with static human-authored rules.

The server can use various training and learning mechanisms to improve internal models used for clustering and profiling. The server may train supervised or semi-supervised models on historical lifestyle pattern information and ground-truth labels indicating successful recommendation outcomes. The server can use an optimization objective such as cross-entropy loss or mean squared error, performing gradient descent with weight updates implemented using a standard optimizer algorithm. The server may augment training data by synthesizing variations in time ranges, location noise, or spending patterns to make the models more robust to real-world variation. By updating internal models based on continuous streams of usage result information, the server adapts to long-term changes in user behavior and external conditions.

By implementing these hardware and software configurations, the server and terminal together realize more than a generic automation of human planning. The server performs technical data transformations and control operations that exploit high-dimensional behavior and context data to drive the internal configuration and invocation of a generative AI model.

The server uses specialized data structures, clustering algorithms, statistical feature extraction, template-based prompt generation, and outcome-aware optimization to convert raw sensor and transaction data into stable, context-specific prompt sentences. This improves the speed and accuracy of generating relevant lifestyle plan information, decreases computational overhead by avoiding unnecessary or poorly specified calls to the generative AI model, and reduces communication load between the server and external model infrastructure. Consequently, the system improves the functioning of the underlying computer-based recommendation pipeline itself, not merely the efficiency of a business process, by providing a concrete technical solution to managing large-scale user behavior data and controlling generative AI outputs in a feedback-informed manner.

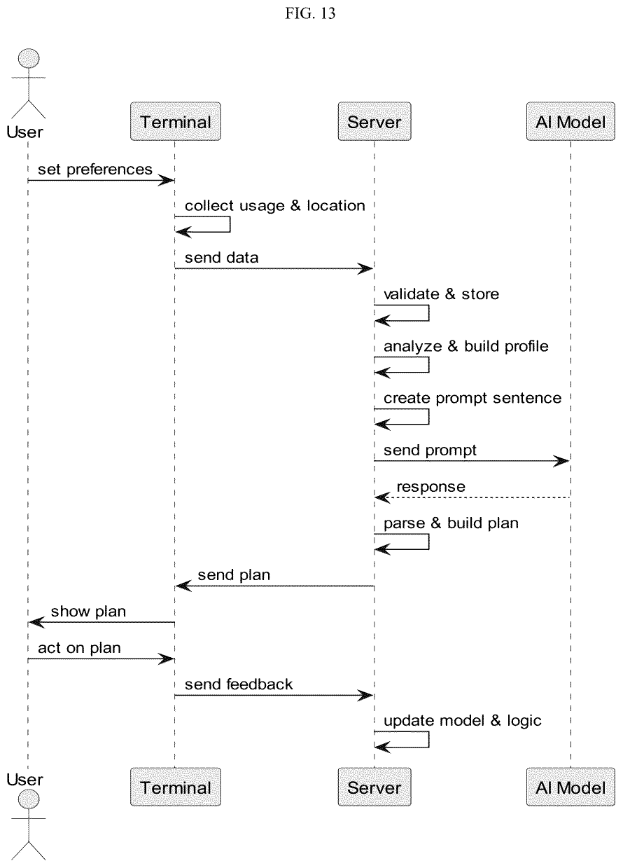

The following describes the processing flow using FIG. 13.

### Step 1:

The user operates the terminal and grants permission for acquisition of usage history information and location information. The input is a user consent state and configuration settings such as allowed data types, sampling intervals, and privacy preferences. The terminal displays configuration screens, receives user selections, and writes the selected parameters into local storage as configuration records. The output is stored configuration data that specify how the terminal will subsequently collect transaction records and location records.

### Step 2:

The terminal acquires location information from the operating system's location service. The input is the configuration data (sampling interval, distance threshold) and sensor signals from components such as GPS, Wi-Fi positioning, or cellular triangulation. The terminal calls a location API, receives latitude, longitude, timestamp, and accuracy values, and converts them into a normalized coordinate representation. The terminal stores the normalized coordinates and associated metadata in a local structured format, such as a row in a local database table. The output is a set of locally stored location records with standardized fields.

### Step 3:

The terminal acquires usage history information from one or more service applications. The input is the configuration data and transaction feeds obtained from an application programming interface exposed by a payment service or service provider. The terminal issues API requests that include a user identifier and a time range, receives transaction records including activity type, amount, merchant category, and timestamp, and converts returned payloads into internal data structures. The terminal filters out duplicate or incomplete records based on identifiers and timestamps. The output is a cleaned set of local usage history records stored in a local database or similar data store.

### Step 4:

The terminal prepares and transmits the collected data to the server via a secure communication path. The input is the locally stored location records and usage history records and an authentication token associated with the user. The terminal serializes the records into a structured text-based payload, such as JSON, bundles them with metadata including device identifier and protocol version, and opens a Transport Layer Security session with the server. The terminal performs encryption of the payload and attaches the authentication token in a request header. The output is an encrypted network request containing normalized usage history information and location information delivered to the server.

### Step 5:

The server receives and authenticates the request from the terminal. The input is the encrypted network request and the server's cryptographic credentials. The server terminates the TLS session, decrypts the payload, and validates the authentication token by checking its signature, issuer, and expiration time. The server also validates protocol version and device identifier fields. If validation fails, the server generates an error log and discards the payload. If validation succeeds, the server passes the decrypted payload to subsequent processing modules. The output is a verified and deserialized payload containing raw usage history data and raw location data in a server-internal representation.

### Step 6:

The server validates and normalizes the data fields contained in the payload. The input is the deserialized usage history data and location data. The server checks each record for required fields, ensures that timestamps conform to a defined time format, ensures that monetary amounts are numeric and within configured ranges, and checks that latitude and longitude values fall within valid geographic bounds. The server corrects minor format inconsistencies, rejects records that fail critical checks, and assigns error codes to problematic entries. The server converts acceptable records into a normalized schema by mapping field names and data types to the server's standard internal specification. The output is a set of normalized and validated records ready for database insertion.

### Step 7:

The server stores the normalized records into a relational database. The input is the normalized usage history records and normalized location records. The server opens a connection to a database engine, constructs parameterized SQL INSERT statements for transaction tables and location tables, and executes these statements in batched form to reduce overhead. The server uses indexes on fields such as user_id and timestamp to enable efficient retrieval later, and commits the transaction to make the data durable. The output is a persistent database state in which the new records are stored in “transactions” and “location_data” tables with consistent schemas.

### Step 8:

The server retrieves a relevant subset of stored data for analysis. The input is a user identifier, an analysis time window, and optional filter conditions such as location region or transaction category. The server executes SQL SELECT queries that extract transaction rows and location rows for the specified user within the time window. The server loads the result sets into an in-memory analytical structure similar to a DataFrame. The server converts timestamp strings into high-precision time types and ensures that numeric fields such as amounts and coordinates use appropriate numeric types. The output is a pair of in-memory datasets containing structured transaction data and structured location data for the selected analysis period.

### Step 9:

The server analyzes the transaction data to derive spending-related features. The input is the in-memory transaction dataset. The server performs grouping operations over dimensions such as merchant category, day of week, and time of day to compute aggregated statistics including frequency of transactions, total spending, average spending per transaction, and variance of spending. The server also identifies top-N categories by frequency and amount, detects periodic patterns such as weekly or monthly cycles, and flags anomalous spikes in spending. The server encodes these results into feature vectors that describe the user's consumption characteristics. The output is a structured set of transaction-based features representing expenditure tendencies.

### Step 10:

The server analyzes the location data to derive mobility-related features. The input is the in-memory location dataset. The server cleans the data by removing outliers based on impossible speeds or isolated single-point anomalies. The server applies a clustering algorithm to the latitude and longitude fields, such as k-means or density-based clustering, using configurable parameters like number of clusters, distance threshold, and minimum samples. The server assigns each location record to a cluster identifier, then computes for each cluster the visit count, total dwell time, average arrival time, and typical days of the week. The server identifies clusters that likely correspond to home, workplace, and recurring leisure areas based on temporal distribution patterns. The output is a structured set of location-based features representing movement tendencies and frequent areas.

### Step 11:

The server fuses transaction-based features and location-based features to construct lifestyle pattern information. The input is the transaction-based feature set and the location-based feature set. The server matches transactions to clusters by comparing timestamps and, where possible, spatial proximity between transaction locations and cluster centroids. The server computes statistics such as spending per cluster, categories dominant in each cluster, and time-of-day distributions at each cluster. The server identifies joint patterns such as “frequent lunch spending at a specific cluster on weekdays” or “recurrent cafe visits near home on weekend afternoons.” The server encodes these joint patterns into a machine-readable structure containing fields for frequent_areas, preferred_time_slots, preferred_price_ranges, and spending_hotspots. The output is lifestyle pattern information summarizing combined behavior and movement characteristics.

### Step 12:

The server generates profile information from the lifestyle pattern information. The input is the lifestyle pattern information. The server applies rule-based logic and thresholding to categorize continuous features into discrete tendencies. For example, the server classifies an area as a preferred lunch area if spending frequency and consistency across weeks exceed configured thresholds, and classifies a price range as preferred if the majority of spending events fall within that price range. The server also derives preference tendencies from recurrent categories and locations. The server stores the resulting profile information in a profile storage structure indexed by user identifier. The output is profile information summarizing behavioral tendencies, movement tendencies, expenditure tendencies, and preference tendencies of the user.

### Step 13:

The server determines the context and objective for recommendation generation. The input is the profile information, current time and date, environment information such as weather or special events, and optionally an explicit user preference setting such as a goal to save money or to explore new facilities. The server interprets this input using decision rules to select a recommendation scenario, such as weekday lunch, weekend leisure, or spending reduction. The server outputs a scenario type and a set of constraints and goals, such as geographical radius, budget level, and number of recommended items, to be used in prompt construction.

### Step 14:

The server constructs a prompt sentence for a generative AI model based on the scenario and profile information. The input is the scenario type, constraints and goals, and the profile information. The server selects a template corresponding to the scenario from a template library and fills the template with concrete values such as area names, time ranges, preferred categories, and budget constraints. The server may, for example, generate a prompt sentence such as “The user usually has lunch near the central business area between 11:30 and 13:30 on weekdays and prefers moderately priced casual restaurants. Based on this pattern, please propose three optimal lunch spots for this week. Include each restaurant's name, type of cuisine, approximate price range, and a short explanation of why it matches the user's preferences.” The output is a textual prompt sentence that encodes the user's lifestyle profile and current objective in natural language.

### Step 15:

The server transmits the prompt sentence to the generative AI model and requests a response. The input is the prompt sentence and configuration parameters such as maximum output length, sampling temperature, and desired output style. The server uses an interface program to call a local or remote generative AI model endpoint, sending the prompt and parameters through an HTTP or similar protocol. The generative AI model tokenizes the prompt, processes it through its neural network layers, and produces a text response. The server receives the response payload at the interface program. The output is response information from the generative AI model containing one or more lifestyle plan candidates expressed in natural language.

### Step 16:

The server parses and structures the response information into lifestyle plan information. The input is the response text from the generative AI model. The server identifies individual recommendation items by detecting enumerated lists, line breaks, or punctuation-based separators. The server extracts fields such as recommended facility name, activity description, suggested time slot, and price range through pattern matching or simple semantic parsing. The server optionally calls an external geocoding service to resolve facility names to coordinates and checks whether recommendations conflict with hard constraints, such as exceeding the budget or being outside the allowed distance. The server then assembles the extracted and validated data into structured lifestyle plan records. The output is lifestyle plan information containing candidate actions, candidate facilities, and candidate time slots, formatted in a structured representation.

### Step 17:

The server delivers the lifestyle plan information to the terminal for presentation. The input is the lifestyle plan information and identification of the target user terminal. The server decides whether to push a notification or wait for a pull request based on configuration. The server encapsulates the lifestyle plan information in a compact structured payload and sends it via a push messaging service or via a direct HTTP response to a terminal request. The output is a transmitted dataset that the terminal can render to the user.

### Step 18:

The terminal receives the lifestyle plan information and displays it to the user. The input is the payload containing the lifestyle plan information. The terminal parses the structured records into in-app objects, sorts or filters recommendations as needed, and renders them on the display with text labels, icons, and optionally map views using a map software development kit. The terminal enables interactive actions such as selecting a recommendation for more details, initiating navigation, or marking a recommendation as not interested. The output is a visual and interactive presentation of lifestyle plan information to the user.

### Step 19:

The user reviews the recommendations and performs follow-up actions. The input is the lifestyle plan information as presented on the terminal display. The user may choose to visit a recommended facility, schedule an activity, or ignore certain recommendations. The user may also adjust preferences, such as tightening a budget constraint or changing a preferred area, by operating the application interface. The output is actual user behavior and updated preference settings, which are implicitly or explicitly captured as new usage history information and location information.

### Step 20:

The terminal captures usage result information and sends it back to the server for feedback processing. The input is the new usage history information (transactions and interactions) and new location information that occur after the lifestyle plan was presented. The terminal aggregates these records and links them to the corresponding plan identifier when available. The terminal serializes and encrypts the data and transmits them to the server in the same manner as initial data. The output is a new batch of feedback data delivered to the server.

### Step 21:

The server incorporates the usage result information into its database and updates internal models and prompt generation logic. The input is the new feedback data, including which recommendations were followed and which were ignored. The server stores the data in feedback-related tables and joins this information with historical recommendation logs. The server computes statistics such as follow-through rate per scenario, acceptance rate for particular categories or areas, and correlation between prompt structure and user response. The server then adjusts cluster thresholds, preference weights, and template selection rules, and may update model parameters for internal classifiers. The output is an updated set of lifestyle pattern information and generation logic for prompt sentences that improves future recommendation precision and computational efficiency.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional recommendation systems and lifestyle planning systems typically process limited categories of user data (such as simple preference tags or past purchases) in isolated pipelines. These systems often rely on static rules or simple scoring functions executed on generic processors, without deeply integrating heterogeneous data streams such as real-time emotional state, fine-grained behavioral logs, environmental conditions, and predicted future tendencies. As a result, the generated recommendations are coarse, context-insensitive, and frequently misaligned with the user's current situation, thereby reducing user engagement and utility.

From a computer technology standpoint, existing architectures do not provide an integrated data-processing and control mechanism that (i) dynamically fuses multi-modal user information (attributes, behavior, emotion) and multi-source external information (weather, environment, reference data), (ii) automatically generates prompt sentences for a generative AI model in a machine-optimizable and feedback-adaptable manner, and (iii) orchestrates downstream plan generation and adjustment in a closed loop. Known systems typically treat a generative AI model as a black-box text generator invoked with manually crafted prompts, which are not systematically constructed from machine-learned feature sets nor iteratively refined based on user feedback in a structured way.

Furthermore, conventional systems do not effectively exploit machine learning preprocessing pipelines on multi-modal data to predict future purchase behavior, movement tendencies, and emotional tendencies, and do not tightly couple such predictive outputs to the construction of input prompt sentences. As a consequence, the underlying computing resources are not used efficiently: redundant data is processed repeatedly, prompts are suboptimal or inconsistent, and the resulting plans do not reflect the most recent model state or feedback. There is thus a need for a computer-implemented technique that improves the way a processor acquires, normalizes, fuses, and exploits multi-modal data; constructs prompt sentences for generative AI models; and iteratively refines internal data structures and prompt construction logic based on feedback, thereby improving the operation of the computing system itself rather than merely automating a human mental process.

The specific processing by the specific processing unit 290 of the data processing device 12 in Application Example 2 is realized by the following means.

The present invention provides a server comprising a processor configured to acquire user-related information including preference information, health condition information, food preference information, exercise-related information, and location-related information as electronic user attribute information; to acquire purchase history information and position information of a user as electronic behavioral history information; to acquire expression information, voice information, and text information of the user, to estimate an emotional state of the user based on the acquired information, and to store the estimated emotional state as electronic emotional information; to acquire weather information, life-related information, and research-related information from an external information providing apparatus as electronic external environment information; to perform, by machine learning processing, preprocessing on the user attribute information, the behavioral history information, the emotional information, and the external environment information so as to generate a set of feature quantities and to predict, based on the set of feature quantities, at least a future purchase behavior, a movement tendency, and an emotional tendency of the user; to construct, in a natural language, an instruction sentence for input to a generative AI model based on the set of feature quantities and a prediction result and to generate a prompt sentence including the instruction sentence; to input the prompt sentence and the set of feature quantities to the generative AI model and to cause the generative AI model to generate plan information including at least daily life plan information, expenditure suppression plan information, meal plan information, purchase plan information, and facility utilization plan information; to adjust the plan information so as to incorporate at least one of a stress-relief activity, an emotional-improvement activity, and a rest activity into the plan information based on the emotional information and the emotional tendency; to extract, based on the position information and the weather information, at least one actual commercial facility and outdoor facility corresponding to activities included in the plan information and to expand the plan information as experience plan information according to a current position of the user; and to present the adjusted plan information and the experience plan information to a user terminal in a form including time-zone-specific action instructions, cost estimation information, and movement route information and to acquire an operation result of the user as feedback information and update the set of feature quantities and conditions for constructing the instruction sentence based on the feedback information. This enables an improved computer-implemented pipeline in which heterogeneous user and environmental data are normalized and fused into machine-learned feature sets, in which prompt sentences for a generative AI model are automatically and adaptively constructed from those feature sets, and in which the processor operates a closed-loop control mechanism that refines both internal models and prompt construction logic based on user feedback, thereby enhancing the efficiency, accuracy, and contextual relevance of plan generation performed by the computing system.

The term “user-related information” refers to information associated with an individual who uses the system, including but not limited to preference information, health condition information, food preference information, exercise-related information, and location-related information.

The term “preference information” refers to information indicating a tendency, liking, or disliking of a user with respect to activities, items, services, or environments.

The term “health condition information” refers to information indicating a physical or mental state of a user, including at least indicators of wellness, fatigue, stress, or medical constraints relevant to lifestyle planning.

The term “food preference information” refers to information indicating types of food, ingredients, or meals that a user prefers, avoids, or is restricted from consuming.

The term “exercise-related information” refers to information indicating kinds, frequency, intensity, or preferences of physical activities or sports practiced or desired by a user.

The term “location-related information” refers to information indicating a geographic position or movement of a user, including at least current position, historical positions, or frequently visited places.

The term “user attribute information” refers to electronic data representing user-related information that has been acquired, normalized, and stored as structured information for processing by the system.

The term “purchase history information” refers to information indicating past economic transactions of a user, including at least transaction dates, amounts, merchant identifiers, and product or service categories.

The term “position information” refers to information specifying a location of a user or a user device, typically represented by coordinates, address data, or place identifiers.

The term “behavioral history information” refers to electronic data indicating past actions or activities of a user, including at least purchase history information and position information, and optionally application usage logs or visit histories.

The term “expression information” refers to information derived from visual data of a user's face, including facial features or expressions captured by an image-capturing device.

The term “voice information” refers to information derived from acoustic data of a user's speech, including at least tone, pitch, intensity, and temporal patterns of the voice signal.

The term “text information” refers to character-based or symbol-based content generated or provided by a user, including at least messages, comments, or input sentences expressing mood, status, or preferences.

The term “emotional state” refers to a psychological condition of a user at a point in time, including but not limited to states such as joy, sadness, stress, calmness, or fatigue.

The term “emotional information” refers to electronic data representing an estimated emotional state of a user, including at least labels, scores, or vectors indicating emotional categories and intensities.

The term “weather information” refers to information indicating meteorological conditions at a location, including at least temperature, precipitation, wind, and descriptive weather status.

The term “life-related information” refers to information related to general living conditions or lifestyle factors, such as local events, facility information, or typical activity patterns relevant to daily life.

The term “research-related information” refers to information derived from external studies, statistics, or reports, including at least aggregated health guidelines, behavioral norms, or lifestyle recommendations.

The term “external information providing apparatus” refers to an external system or service that supplies weather information, life-related information, research-related information, or similar data via a communication network.

The term “external environment information” refers to electronic data representing conditions external to the user, including at least weather information, life-related information, and research-related information.

The term “machine learning processing” refers to processing using an algorithm or model that learns patterns from data, including at least classification, regression, clustering, or feature extraction methods executed by a processor.

The term “preprocessing” refers to operations applied to raw data prior to higher-level analysis, including at least cleaning, normalization, feature generation, aggregation, and fusion of heterogeneous data.

The term “set of feature quantities” refers to a collection of numerical or categorical variables derived from user attribute information, behavioral history information, emotional information, and external environment information, and used as input to predictive models or other algorithms.

The term “future purchase behavior” refers to a predicted tendency of a user to perform specific economic transactions in a forthcoming time period, including likelihoods of purchasing certain categories or amounts.

The term “movement tendency” refers to a predicted pattern of user movement or location visits in a forthcoming time period, including frequencies or probabilities of visiting certain types of places or areas.

The term “emotional tendency” refers to a predicted pattern or trajectory of a user's emotional state over time, including tendencies toward stress, relaxation, or other emotional conditions.

The term “instruction sentence” refers to a natural language sentence that specifies a processing request or task description to be provided as part of an input to a generative AI model.

The term “prompt sentence” refers to a natural language expression, including at least one instruction sentence and optionally additional context, that is input to a generative AI model to cause generation of output content.

The term “generative AI model” refers to an information processing model, implemented by an artificial intelligence technique, that generates output data such as text, plans, or structured content in response to an input prompt sentence and associated context.

The term “plan information” refers to electronic data representing at least one proposed course of action or schedule for a user, including daily life plan information, expenditure suppression plan information, meal plan information, purchase plan information, and facility utilization plan information.

The term “daily life plan information” refers to plan information that proposes time-ordered daily activities for a user, such as work, exercise, rest, and leisure activities.

The term “expenditure suppression plan information” refers to plan information that proposes actions or schedules designed to reduce or optimize monetary spending by a user while maintaining desired living conditions.

The term “meal plan information” refers to plan information that proposes meals, food items, or dietary patterns for a user, taking into account food preference information and health condition information.

The term “purchase plan information” refers to plan information that proposes purchasing activities for goods or services, including timing, categories, and locations of purchases.

The term “facility utilization plan information” refers to plan information that proposes use of physical facilities, such as commercial facilities, service facilities, or outdoor facilities, in connection with scheduled activities.

The term “stress-relief activity” refers to an activity proposed to alleviate or reduce stress of a user, such as relaxation, leisure, or rest-oriented actions.

The term “emotional-improvement activity” refers to an activity proposed to improve a user's emotional state, for example by increasing positive emotions or reducing negative emotions.

The term “rest activity” refers to an activity proposed to provide physical or mental rest for a user, such as sleep, short breaks, or low-effort recreation.

The term “commercial facility” refers to a physical place where goods or services are provided to customers, such as a store, shop, restaurant, or service establishment.

The term “outdoor facility” refers to a physical place primarily used for outdoor activities, such as parks, sports grounds, or open recreational areas.

The term “experience plan information” refers to plan information that has been expanded to include specific commercial facilities or outdoor facilities, and that describes concrete in-person experiences tailored to a user's current position and context.

The term “time-zone-specific action instructions” refers to instructions that associate proposed user actions with particular time ranges within a day, such as morning, afternoon, evening, or specific time slots.

The term “cost estimation information” refers to information indicating an estimated monetary cost associated with at least part of the plan information, including expenses for facilities, services, or purchases.

The term “movement route information” refers to information indicating an ordered sequence of locations or waypoints that a user is recommended to move along, such as navigation paths between facilities.

The term “user terminal” refers to an information processing device operated by a user, such as a portable communication device, computing device, or display device, that presents plan information and receives user input.

The term “feedback information” refers to information indicating a user's response or interaction with presented plan information, including at least acceptance, modification, rejection, or execution of recommended activities.

The term “conditions for constructing the instruction sentence” refers to rules, parameters, or templates used by the processor to determine content, structure, and wording of the instruction sentence included in the prompt sentence.

In one embodiment, a server cooperates with one or more terminals operated by a user to implement the claimed system. The server includes at least one processor, a memory storing program instructions, and a communication interface. The terminal includes a processor, a display, one or more input devices, and optionally a camera, a microphone, a positioning module, and a wireless communication module. The server executes server-side software including an operating system, a database management system, a web application framework, a machine learning library, and a generative AI module. The terminal executes client-side software providing a graphical user interface and sensor access.

The server uses a database system such as a relational database management system to store user-related information, behavioral history information, emotional information, and external environment information. The server uses a data-processing library such as a tabular-data processing framework to perform data cleaning, aggregation, and feature extraction. The server uses a machine learning library such as a general-purpose machine learning toolkit to implement prediction models on structured features. The server uses a neural network framework such as a deep-learning framework to implement a generative AI model based on a neural network architecture.

The terminal acquires user-related information by displaying screens that prompt the user to input preference information, health condition information, food preference information, exercise-related information, and location-related information. The terminal sends the user input to the server as structured messages, for example, in a key-value representation. The terminal further acquires expression information by capturing images or video sequences of the user's face using the camera, voice information by recording audio using the microphone, and text information by accepting typed comments or selections from the user. The terminal transmits these data to the server via a network.

The server stores the received user-related information as user attribute information in a structured form such as rows in multiple relational tables or fields in a document store. The server assigns each user a unique identifier and associates the user attribute information with this identifier. The server stores purchase history information, received for example from financial transaction interfaces, and position information, received from the terminal or from a location service, as behavioral history information. The server maintains behavioral history information in time-stamped records indexed by user identifier and time.

The server estimates an emotional state of the user by processing expression information, voice information, and text information. In one embodiment, the server uses a convolutional neural network (CNN) implemented in the deep-learning framework to process facial images. The CNN includes multiple convolutional layers, pooling layers, and fully connected layers.

The server trains the CNN on labeled facial-expression datasets using a loss function such as cross-entropy and an optimization method such as stochastic gradient descent or Adam. The server extracts an emotional label and associated probabilities from the CNN output.

The server analyzes voice information by applying a sequence model such as a recurrent neural network, a long short-term memory network, or a transformer-based audio classifier to features such as Mel-frequency cepstral coefficients. The server trains the audio model in a similar manner using labeled speech-emotion datasets. The server analyzes text information by applying a natural-language model such as a transformer encoder to the text tokens, and derives sentiment scores or emotion categories. The server fuses the results from the image model, audio model, and text model using a fusion algorithm such as weighted averaging or a small fully connected network that receives the individual emotion vectors as input and outputs a combined emotional-state vector. The server stores this combined emotional-state vector as emotional information, together with a time stamp and the user identifier.

The server acquires external environment information by calling network-based information sources. For example, the server acquires weather information by sending a request containing a location parameter and an authentication token to a weather information service and receiving meteorological data including temperature, precipitation probability, wind speed, and weather description. The server stores this meteorological data as weather information in external environment information tables. The server acquires life-related information or research-related information from other data services, such as health guideline repositories or event databases, and stores them as external environment information.

The server preprocesses user attribute information, behavioral history information, emotional information, and external environment information to generate a set of feature quantities. The server uses the tabular-data processing framework to join, resample, and transform these data. For example, the server computes average daily spending, counts of visits to particular place categories, average emotion scores over time windows, and simple indicators derived from weather information such as flags indicating good conditions for outdoor activities. The server organizes these derived values into feature vectors for each user and for each time segment.

The server applies machine learning processing to these feature vectors in order to predict future purchase behavior, movement tendencies, and emotional tendencies. In one example, the server uses a gradient-boosted decision tree model to predict probabilities that the user will purchase goods in a particular category in the next week. The server trains this model using historical feature vectors as input and past purchases as labels. The server uses a clustering algorithm such as k-means or a density-based method to identify clusters of movement patterns and assigns a cluster label representing movement tendency to each user. The server uses a time-series prediction model, such as a recurrent neural network, to estimate future emotional tendencies based on sequences of past emotional states and contextual features.

The server constructs an instruction sentence in natural language for input to a generative AI model based on the set of feature quantities and one or more prediction results. The server uses a template-based generator, where fixed parts of the sentence are combined with values or descriptors derived from the features and predictions. The server may store different templates depending on the type of plan to be generated, such as daily life plan, expenditure suppression plan, meal plan, purchase plan, or facility utilization plan. The server then constructs a prompt sentence including the instruction sentence and additional context sentences describing user attributes, predictions, and external environment conditions. For example, the server may generate a prompt sentence such as:
“The user's hobby is running, the health status is good, and the weather today is sunny with moderate temperature. Please generate a one-day lifestyle plan including a morning run, appropriate meals, and evening relaxation.”

In another example, the server may generate:
“The user is currently feeling stressed and often spends money late at night at cafés near the office. Using the user's past six months of transaction history and location patterns, please propose a one-week plan to reduce stress and unnecessary spending.”

In another example, the server may generate:
“The user's hobby is hiking, and the recent emotional state is tired. The weekend weather forecast is clear. Please propose a relaxing hiking course, appropriate break times, and post-hike recovery activities.”

In yet another example, the server may generate:
“The user is feeling tired and prefers light, non-spicy food. Considering past order history and health data, please generate today's optimal meal plan that supports recovery and stress reduction.”

The server inputs the constructed prompt sentence and, in some embodiments, the set of feature quantities encoded as additional context into a generative AI model. The generative AI model is implemented as a neural network such as a transformer-based language model. The model includes an embedding layer, multiple self-attention layers, feedforward sub-layers, and a final output projection layer. The server trains or fine-tunes the generative AI model on training data composed of input prompt sentences and target plan descriptions. During training, the server computes a loss such as cross-entropy over target tokens and updates the model parameters using gradient-based optimization. The server may perform regularization techniques such as dropout and data augmentation on text to improve generalization.

The server uses the trained generative AI model to generate plan information by performing inference given the prompt sentence and optional structured context. The generative AI model outputs text sequences representing the plan information. The server parses the output into structured fields such as daily life plan information, expenditure suppression plan information, meal plan information, purchase plan information, and facility utilization plan information. The server may impose decoding constraints during generation, such as limiting maximum length or enforcing section markers, to produce machine-parsable output.

The server adjusts the plan information based on emotional information and emotional tendency. For example, when emotional information indicates a high stress level, the server modifies the plan information by increasing the number or duration of stress-relief activities, such as relaxation sessions or low-intensity outdoor walks. The server may apply heuristic rules or a secondary optimization module that reweights candidate activities according to emotional scores and predicted emotional trajectory. The server thereby incorporates at least one of a stress-relief activity, an emotional-improvement activity, and a rest activity into the plan.

The server uses position information and weather information to select specific facilities corresponding to proposed activities in the plan. The server accesses a facility database or an external mapping service to retrieve candidate commercial facilities and outdoor facilities within a given radius of the user's current position or expected movement path. The server computes distances and estimated travel times to each facility. The server then binds activities in the plan to particular facilities that satisfy constraints such as distance thresholds, activity type, and weather suitability. The server thereby expands the plan information into experience plan information that includes concrete locations and sequences of visits.

The server presents the adjusted plan information and experience plan information to the terminal. The server formats the information into structures that indicate time-zone-specific action instructions, estimated costs, and movement route information. The server may compress large plan data or images to reduce communication load. The terminal receives this information and renders it on the display in human-readable form. For example, the terminal shows a day schedule divided into morning, afternoon, and evening, with each block listing recommended activities, estimated costs, and buttons that open navigation or reservation functions.

The user reviews the presented plan and interacts with the terminal to accept, modify, or reject specific items. The terminal sends these interaction results as feedback information to the server. The server logs which recommendations the user accepted, which activities the user performed or skipped, and any manual adjustments the user made. The server incorporates this feedback information into subsequent preprocessing and model updates. For example, the server re-estimates preference weights for activity categories when acceptance rates change. The server retrains or fine-tunes the feature-based prediction models and, in some embodiments, the generative AI model using recent data including feedback. The server also modifies conditions for constructing instruction sentences, such as template selection and weighting of features included in the prompt sentence.

This architecture improves computer technology in several ways. The server eliminates redundant and ad hoc prompt crafting by automatically generating prompt sentences from structured feature quantities derived by machine learning preprocessing. Because the prompt construction logic uses learned features and predictions, prompt sentences become more consistent and informative for the generative AI model, improving generation accuracy and reducing unnecessary tokens. This contributes to reduced communication payloads when the generative AI model is deployed on a separate machine or service. The fusion of multi-modal data (user attributes, behavior, emotion, and environment) into compact feature vectors reduces storage and computation requirements in downstream components because the generative model receives summarized context instead of large raw logs.

The server also employs non-conventional processing flows and data structures. For example, the server simultaneously maintains a feature store that aggregates multi-modal signals per user and time window, an emotion log that tracks fused emotion vectors, and a feedback log linked to specific generated plan segments. This design allows the server to update both predictive models and prompt construction rules in a closed loop, which is not a simple automation of human recommendations but a system-level optimization of data pipelines, model inputs, and output consumption. By using specialized machine learning algorithms (decision trees, recurrent networks, transformers) with explicit loss functions and weight-update procedures, the server achieves improved prediction accuracy and plan relevance compared with static rule-based systems.

The integration of emotional-state estimation, weather-aware facility selection, and generative plan synthesis yields technical effects such as more accurate targeting of computational resources. For instance, the server can adapt the length and complexity of generated plans based on predicted user engagement or stress, thereby reducing server load when shorter plans are likely to be effective. The closed-loop adaptation of prompt construction rules based on user feedback ensures that the generative AI model is invoked with more efficient and relevant prompts over time, reducing the number of iterations needed to obtain acceptable outputs and improving overall processing throughput.

In another embodiment, the server is implemented in a distributed fashion across multiple computing nodes. A first node handles data ingestion and preprocessing, a second node hosts prediction models, and a third node hosts the generative AI model. The server coordinates these nodes by passing feature vectors and prompt sentences over an internal network. This division allows each node to be optimized for particular tasks, such as using hardware acceleration for neural-network inference on the generative AI node, thereby improving response times when generating plans.

In yet another embodiment, the terminal performs part of the emotional-state estimation locally, for example by running a lightweight neural network on the terminal processor to extract basic emotion scores from facial images. The terminal sends only compressed emotion vectors to the server rather than raw images, which reduces communication bandwidth and protects user privacy. The server then integrates these emotion vectors into the feature set and performs the remaining processing steps as described above.

In a further embodiment, the system supports different generative AI model architectures and training methods. The server may use an encoder-decoder model for structured plan generation or a decoder-only model pre-trained on generic text and fine-tuned on plan-description corpora. The server may incorporate reinforcement learning from human feedback by adjusting the generative AI model's output distribution based on historical user acceptance of generated plans. The server may further implement curriculum learning or scheduled sampling during training to improve robustness to noisy prompts.

By implementing these embodiments, the server, terminal, and user cooperate in a technical system that not only automates recommendation-like tasks but also improves the way computing resources are used for data acquisition, feature extraction, prompt sentence construction, and neural-network-based plan generation. The specific combination of multi-modal feature fusion, predictive modeling, rule-based and learned prompt construction, and closed-loop feedback adaptation results in enhanced speed, accuracy, and efficiency of the overall computational pipeline, thereby providing a concrete technological improvement beyond a mere abstract scheme.

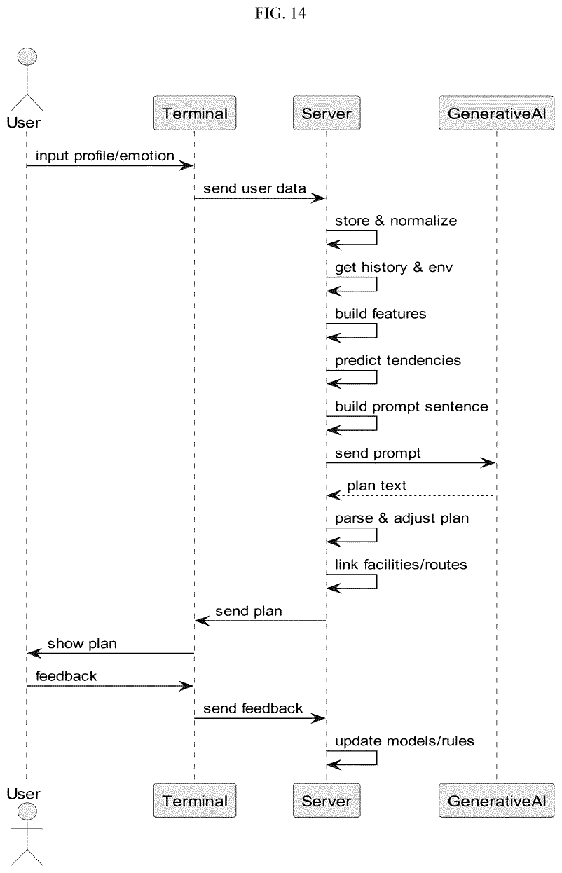

The following describes the processing flow using FIG. 14.

### Step 1:

User inputs profile information into the terminal.

User provides preference information, health condition information, food preference information, exercise-related information, and location-related information through form fields, selection lists, and text boxes displayed on the terminal.

Input: raw user inputs such as selected hobbies, self-reported health status, preferred foods, typical exercises, and home/work locations.

Output: structured profile data objects stored in the terminal (for example, key-value pairs representing each item).

### Step 2:

Terminal transmits user profile information to the server.

Terminal converts the structured profile data into a message format (for example, JSON) and sends the message to a server endpoint over a network connection with user authentication data.

Input: structured profile data stored on the terminal; user authentication token.

Output: a network request containing the profile data, which arrives at the server as a serialized data payload.

### Step 3:

Server stores and normalizes user attribute information.

Server receives the serialized profile payload, validates mandatory fields, converts categorical values into internal codes, and writes the data into user attribute tables in a database management system.

Input: serialized profile payload from the terminal.

Output: normalized user attribute records stored in persistent storage, indexed by user identifier.

### Step 4:

Server acquires purchase history information and position information.

Server calls external transaction interfaces using the user identifier to obtain purchase records and receives transaction details. Server also receives position information from the terminal or from a location service and links it to timestamps.

Input: user identifier, external API credentials, and location messages from the terminal.

Output: behavioral history records containing purchase history information and position information associated with the user.

### Step 5:

Terminal captures expression, voice, and text for emotion estimation.

Terminal activates the camera to capture images or video of the user's face, activates the microphone to record the user's voice, and captures free-text or selected mood labels entered by the user.

Input: sensor signals from camera and microphone, user-typed mood text.

Output: media files (images, audio) and text data stored temporarily on the terminal.

### Step 6:

Terminal sends emotion-related data to the server.

Terminal packages the media files and text into an upload message, applies compression where appropriate, and sends the message to the server using secure communication.

Input: captured image data, audio data, and text data.

Output: a composite request delivered to the server containing raw emotion-related data.

### Step 7:

Server estimates emotional information from multi-modal inputs.

Server processes facial images with a convolutional neural network, processes audio with an audio classifier, and processes text with a language model, then combines the resulting emotion scores into a single emotional-state vector.

Input: image data, audio data, and text data from the terminal.

Data processing: feature extraction (for example, facial features, acoustic features, text embeddings), forward passes through neural networks, and weighted fusion of outputs.

Output: emotional information consisting of emotion labels and intensity scores stored in an emotion log associated with the user.

### Step 8:

Server acquires external environment information.

Server requests weather data and other environment-related data from external services based on the user's location and receives structured responses containing meteorological parameters and life-related information.

Input: user location data and external service identifiers.

Data processing: formation of network requests, parsing of response structures, and mapping of fields to internal representations.

Output: external environment records containing weather information and other environment information stored in a database.

### Step 9:

Server builds a unified feature set from collected data.

Server loads user attribute information, behavioral history information, emotional information, and external environment information for the user, aggregates these data over defined time windows, and computes derived metrics such as averages and counts.

Input: database records for attributes, behavior, emotion, and environment.

Data processing: table joins, time-based grouping, normalization, and computation of feature quantities (for example, average daily spend, visit counts, emotion averages).

Output: a feature vector or set of feature quantities for the user, ready for prediction models.

### Step 10:

Server predicts future tendencies using machine learning models.

Server applies trained prediction models to the feature set to estimate future purchase behavior, movement tendencies, and emotional tendencies by running inference on the feature vector.

Input: feature vector for the user.

Data processing: evaluation of machine learning models such as decision trees or neural networks to compute prediction probabilities or scores.

Output: prediction results describing likely future purchases, movement patterns, and emotional states for the user.

### Step 11:

Server composes an instruction sentence and prompt sentence for a generative AI model.

Server selects a prompt template based on the type of plan to be generated and inserts elements derived from the feature set and prediction results into the template to form a natural-language instruction. Server then adds context sentences to form a complete prompt sentence.

Input: feature set and prediction results for the user, stored prompt templates.

Data processing: template selection, text substitution, and concatenation of instruction and context sentences.

Output: a prompt sentence in natural language specifying tasks for the generative AI model.

### Step 12:

Server optionally supplements the prompt sentence with external context.

Server retrieves recent behavior details or summarized statistics and appends them to the prompt sentence as descriptive text to give the generative AI model more concrete context.

Input: base prompt sentence and recent behavioral summaries.

Data processing: conversion of numerical summaries to descriptive phrases and combination with the existing prompt text.

Output: an extended prompt sentence that includes instruction and supporting context.

### Step 13:

Server sends the prompt sentence and feature set to the generative AI model.

Server encapsulates the prompt sentence and, in some embodiments, encodes the feature set as auxiliary input tokens or key-value context for the generative AI model, and initiates an inference request.

Input: constructed prompt sentence and associated feature quantities.

Data processing: formatting of inputs into the generative AI model's input representation and transmission to the generative AI engine.

Output: an internal call or network request to the generative AI model with the prepared inputs.

### Step 14:

Server receives generated plan information from the generative AI model.

Server obtains generated text representing plan information from the generative AI model and parses it into structured components such as daily life plan, expenditure suppression plan, meal plan, purchase plan, and facility utilization plan.

Input: generated text sequence from the generative AI model.

Data processing: segmentation of text using markers or patterns, extraction of specific sections, and mapping of text segments to structured fields.

Output: structured plan information objects stored in server memory.

### Step 15:

Server adjusts plan information based on emotional information and emotional tendency.

Server analyzes the user's emotional state and predicted emotional trajectory, identifies which parts of the generated plan should be modified, and increases or decreases the presence of stress-relief activities, emotional-improvement activities, and rest activities accordingly.

Input: structured plan information and emotional information for the user.

Data processing: rule-based or learned scoring of each activity, selection or replacement of activities based on emotional criteria, and recalculation of timing or sequencing.

Output: adjusted plan information that is better aligned with the user's current and expected emotional state.

### Step 16:

Server links plan activities to concrete facilities using position and weather information.

Server retrieves facility lists from a facility database or external mapping service, filters facilities that match required activity types, checks distance constraints and weather suitability, and assigns specific locations to the activities in the plan.

Input: adjusted plan information, current position information, and weather information.

Data processing: geographic distance calculation, filtering by category and conditions, and mapping of activities to chosen facilities.

Output: experience plan information containing specific commercial facilities and outdoor facilities associated with plan activities.

### Step 17:

Server generates time-zone-specific action instructions, cost estimates, and route information.

Server organizes the experience plan information into chronological segments (for example, morning, afternoon, evening), estimates monetary costs for each activity, and computes suggested movement routes between facilities using location data.

Input: experience plan information including facilities and scheduled times.

Data processing: time segmentation, cost estimation by summing or averaging expected prices, and route generation by calling routing functions.

Output: a detailed plan representation containing time-zone-specific instructions, cost estimation information, and movement route information.

### Step 18:

Server sends the detailed plan to the terminal.

Server formats the detailed plan into a transmission structure and sends it to the terminal through the communication interface.

Input: detailed plan representation with instructions, costs, and routes.

Data processing: serialization of the plan into a compact format and network transmission.

Output: a response message delivered to the terminal containing the comprehensive plan data.

### Step 19:

Terminal presents the plan to the user via a graphical interface.

Terminal parses the received plan data, displays schedules separated by time zones, lists activities with associated costs and locations, and shows interactive elements for navigation, booking, or modification.

Input: detailed plan data received from the server.

Data processing: transformation of structured plan fields into visual components on the display and association of user-interface controls with corresponding actions.

Output: a visible, interactive plan display presented to the user.

### Step 20:

User interacts with the plan and performs or modifies activities.

User chooses to accept, edit, or reject recommended activities, triggers navigation or booking functions, and optionally records completion of activities through the terminal interface.

Input: plan display and interactive controls shown on the terminal.

Output: interaction events and status updates generated on the terminal corresponding to user choices.

### Step 21:

Terminal transmits feedback information to the server.

Terminal collects the interaction events, such as accepted activities, deleted activities, and changed times, and sends them to the server as feedback records associated with plan items and timestamps.

Input: user interaction events and updated activity states.

Data processing: aggregation of events into feedback objects and transmission to the server.

Output: feedback information messages received by the server.

### Step 22:

Server updates feature sets and prompt construction conditions using feedback.

Server analyzes the feedback information, updates statistical measures of user preferences and acceptance rates, adjusts feature weights or labels, and modifies rules used to build instruction sentences and prompt sentences for future plan generation.

Input: feedback information, existing feature sets, and prompt construction rules.

Data processing: recalculation of preference scores, update of model training data, adjustment of template selection parameters, and storage of new rule parameters.

Output: updated feature sets and revised conditions for constructing prompt sentences, which influence subsequent executions of the plan-generation pipeline.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

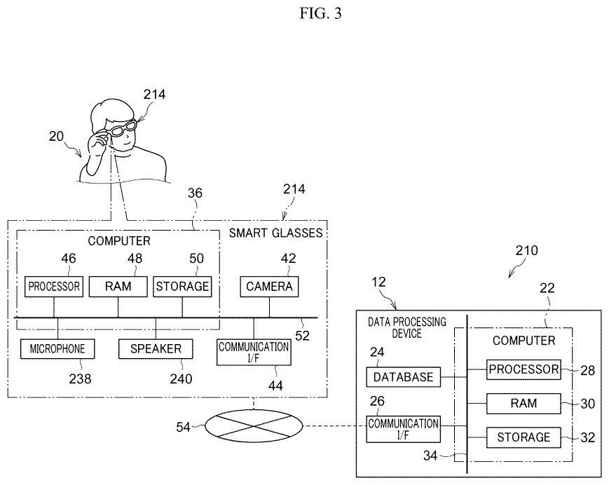

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

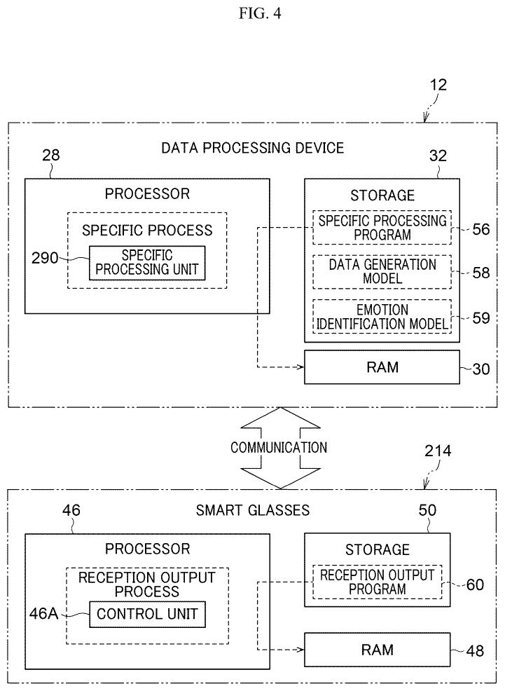

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

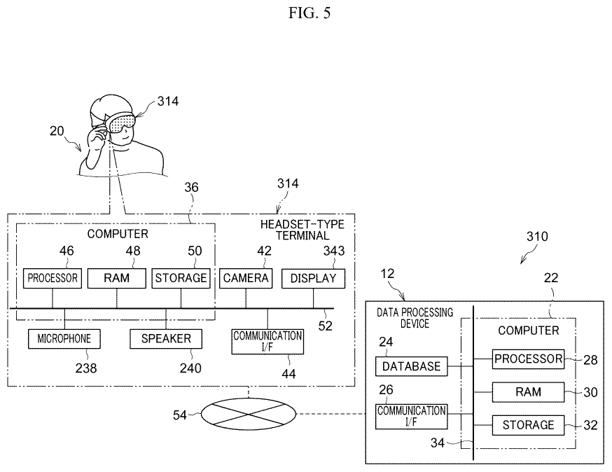

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

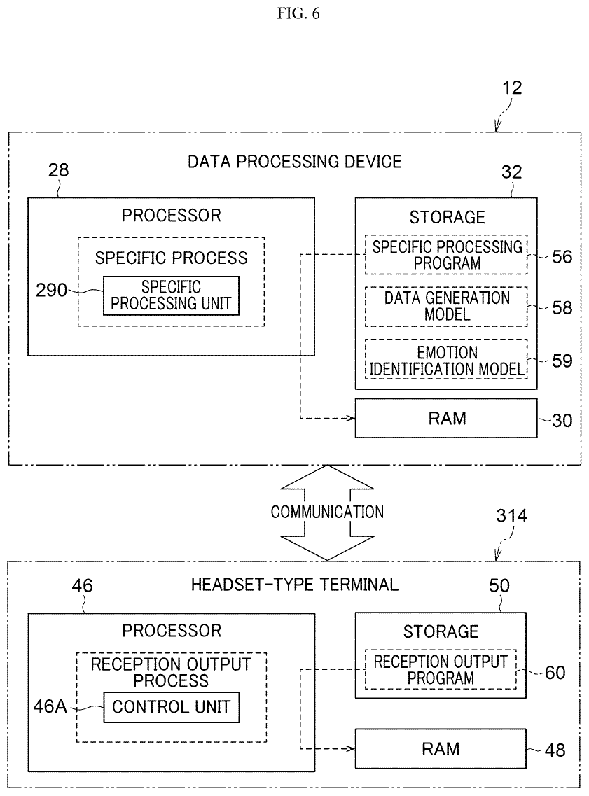

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

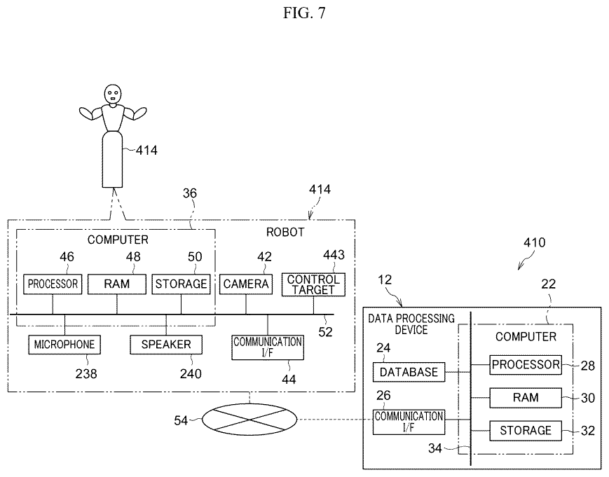

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

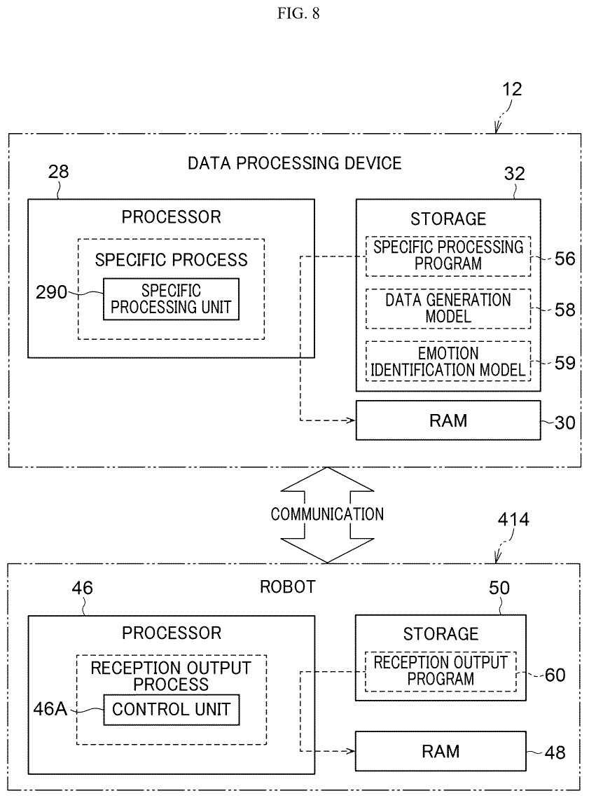

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

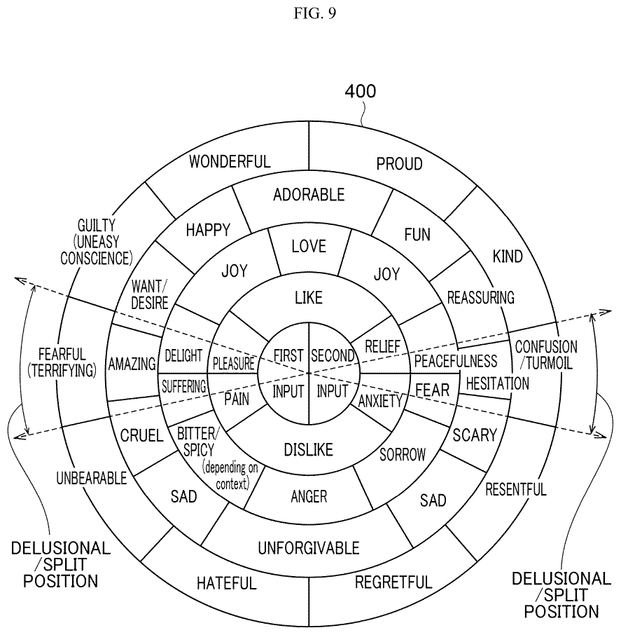

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University).

Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

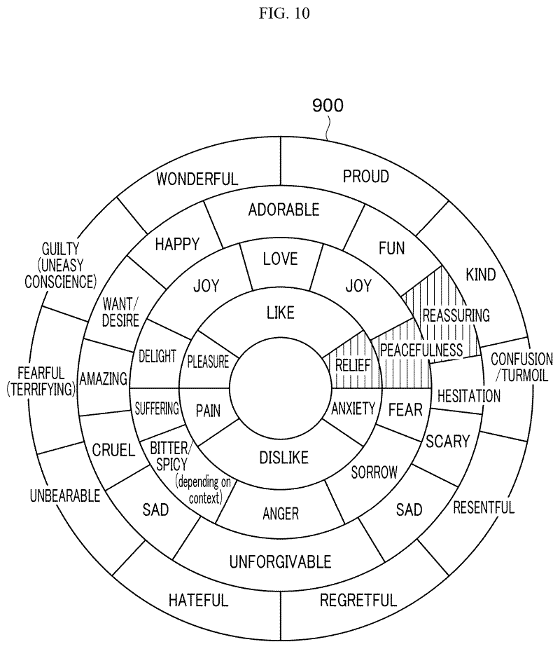

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC).

Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure.

Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor and a storage device,
wherein the processor is configured to
receive, from a terminal operated by a user, information regarding user preferences including hobby information, health condition information, preference information, physical activity information, and location preference information, and convert the received information into electronic information,
obtain, from the terminal or from a data source, history information regarding past life activities of the user, and convert the history information into electronic information, acquire, via a communication network from an external information source, additional information including position information, environment information, and facility information,
acquire the additional information based on position information and behavior history of the user,
integrate the electronic information, the history information, and the additional information into tabular data, and generate feature information from the tabular data, the feature information indicating at least behavior frequency, visit type, time-zone characteristic, and preference tendency of the user,
input the feature information into a machine learning model, estimate behavior patterns and preference tendencies of the user, and calculate evaluation information including at least a health orientation level, an activity orientation level, and a preference intensity level,
combine the evaluation information with the information received from the user to generate profile information including attribute information and behavior characteristic information of the user, and store the profile information in the storage device,
read the profile information from the storage device and generate description information that describes, in a natural language, the attribute information and the behavior characteristic information of the user,
insert the description information into a predetermined instruction template to automatically generate a prompt sentence for input to a generative AI model,
acquire weather information from a weather information source and associate the weather information with the profile information,
generate, based on the profile information and the weather information, a life plan including at least meal content information, physical activity information, leisure activity information, and candidate visit location information, the life plan being structured by time zone,
transmit the prompt sentence to the generative AI model, and obtain, from the generative AI model, response information including natural language text that complements or details content of the life plan, and
integrate the life plan with the response information to generate presentation information for the user, and transmit the presentation information to the terminal so that the presentation information is presented to the user.

### (Supplementary 2)

The system according to supplementary 1,
wherein the processor is configured to
include, in the life plan, meal content candidates, purchasing behavior candidates, and facility usage candidates, and express the meal content candidates, the purchasing behavior candidates, and the facility usage candidates as concrete candidate options based on the natural language text obtained from the generative AI model.

### (Supplementary 3)

The system according to supplementary 1,
wherein the processor is configured to
control the life plan based on the profile information and the evaluation information so as to satisfy constraint conditions regarding expenditure amount and conditions regarding stress reduction, and determine the prompt sentence for input to the generative AI model and content of the life plan such that the user suppresses expenditure while maintaining a lifestyle suitable for the user and achieving stress relief.

### Application Example 1

### (Supplementary 1)

A system comprising a processor,
wherein the processor is configured to
acquire preference information, health information, food-related information, physical-activity-related information, and movement-related information regarding a user, and record the information as structured data, and
record past behavior information and transaction information regarding the user as history data, and
manage an information storage device that stores the structured data and the history data, and extract the structured data and the history data stored in the information storage device, convert the extracted data into numerical data sequences, and perform preprocessing to generate feature quantities for machine learning processing, and
execute computation by using a learned model including a machine learning algorithm with the feature quantities as input, thereby estimating a behavior tendency and a dietary tendency of the user and generating a candidate item sequence to which a suitability degree is assigned for each user, and
apply constraint conditions and operation rules to the candidate item sequence, and generate lifestyle plan information including meal plan information by selecting candidates based on a health condition and a preference condition of the user, and
generate a prompt sentence to be used as input to a generative information processing model, based on the lifestyle plan information and attribute information regarding the user, and input the prompt sentence and the lifestyle plan information into an external generative information processing model, and acquire a generation result including explanation information and additional proposal information corresponding to the lifestyle plan information, and
acquire weather information from an environmental information source, and reflect the weather information in generation of the lifestyle plan information and in creation of the prompt sentence, and
transmit the lifestyle plan information and the generation result to a user information terminal, and cause the user information terminal to present the lifestyle plan information and the generation result visually, and
acquire selection information and evaluation information regarding the lifestyle plan information from the user information terminal, record the selection information and the evaluation information as part of the history data in the information storage device, and reflect the selection information and the evaluation information in learning of the learned model or in generation of the feature quantities.

### (Supplementary 2)

The system according to supplementary 1,
wherein the processor is configured to
generate the lifestyle plan information so as to include the meal plan information, purchase plan information, and facility-visit candidate information.

### (Supplementary 3)

The system according to supplementary 1,
wherein the processor is configured to
generate the lifestyle plan information in consideration of constraint conditions relating to an expenditure amount of the user and by including a candidate item sequence relating to leisure activities, thereby reducing a psychological load of the user.

### Example 2

### (Supplementary 1)

A system comprising a processor,
wherein the processor is configured to
acquire and digitize usage history information and location information related to a user, securely transmit the usage history information and the location information to an information processing apparatus via a communication path,
structure the usage history information and the location information in the information processing apparatus and store the structured information in a storage unit by using an information management mechanism,
aggregate and statistically process, by using an information processing program, the usage history information and the location information stored in the information management mechanism so as to extract lifestyle pattern information including behavioral characteristics and consumption characteristics of the user,
integrate the lifestyle pattern information with time information and environment information so as to generate profile information summarizing behavioral tendencies, movement tendencies, expenditure tendencies, and preference tendencies of the user,
dynamically generate, on the basis of the profile information and the environment information, a prompt sentence to be input to a generative information processing model, input the prompt sentence to the generative information processing model and acquire response information including a lifestyle plan candidate,
extract, from the response information, candidate actions, candidate facilities, and candidate time slots, and generate lifestyle plan information that conforms to the lifestyle pattern information of the user and constraint conditions,
notify and present the lifestyle plan information to a user terminal, and acquire usage result information from the user as the usage history information and the location information again, and update the lifestyle pattern information and a generation process of the prompt sentence.

### (Supplementary 2)

The system according to supplementary 1,
wherein the processor is configured to
include, in the lifestyle plan information, recommendation contents relating to eating and drinking behavior, purchasing behavior, and facility selection.

### (Supplementary 3)

The system according to supplementary 1,
wherein the processor is configured to
optimize the lifestyle plan information, on the basis of the response information from the generative information processing model and the lifestyle pattern information, so as to achieve expenditure suppression and psychological burden reduction while maintaining a lifestyle suitable for the user.

### Application Example 2

### (Supplementary 1)

A system comprising a processor,
wherein the processor is configured to
acquire user-related information including preference information, health condition information, food preference information, exercise-related information, and location-related information, and store the acquired information as electronic user attribute information, and acquire purchase history information and position information of the user and store the acquired information as electronic behavioral history information, and
acquire expression information, voice information, and text information of the user, estimate an emotional state of the user based on the acquired information, and store the estimated emotional state as electronic emotional information, and
acquire weather information, life-related information, and research-related information from an external information providing apparatus and store the acquired information as electronic external environment information, and
perform preprocessing by machine learning processing on the user attribute information, the behavioral history information, the emotional information, and the external environment information so as to generate a set of feature quantities, and predict at least a future purchase behavior, a movement tendency, and an emotional tendency of the user based on the set of feature quantities, and
construct, in a natural language, an instruction sentence for input to a generative AI model based on the set of feature quantities and the predicted result, and generate a prompt sentence including the instruction sentence, and
input the prompt sentence and the set of feature quantities to the generative AI model, and cause the generative AI model to generate plan information including at least daily life plan information, expenditure suppression plan information, meal plan information, purchase plan information, and facility utilization plan information, and
adjust the plan information so as to incorporate at least one of a stress-relief activity, an emotional-improvement activity, and a rest activity into the plan information based on the emotional information and the emotional tendency, and
extract, based on the position information and the weather information, at least one actual commercial facility and outdoor facility corresponding to activities included in the plan information, and expand the plan information as experience plan information according to a current position of the user, and
present the adjusted plan information and the experience plan information to a user terminal in a form including time-zone-specific action instructions, cost estimation information, and movement route information, and acquire an operation result of the user as feedback information and update the set of feature quantities and conditions for constructing the instruction sentence based on the feedback information.

### (Supplementary 2)

The system according to supplementary 1,
wherein the processor is configured to
cause the plan information to include meal recommendation information generated based on the food preference information, the health condition information, and the emotional information of the user, saving recommendation information generated based on an expenditure analysis result and the purchase history information, and in-store utilization recommendation information generated based on the position information and the weather information.

### (Supplementary 3)

The system according to supplementary 1,
wherein the processor is configured to sequentially update the machine learning processing and rules for constructing the prompt sentence to be input to the generative AI model based on the feedback information, and to progressively improve and present the daily life plan information adapted to a behavior pattern, an expenditure tendency, and an emotional tendency of each user.

What is claimed is:

1. A system comprising:
circuitry configured to:
receive, via a communication interface coupled to a packet-switched network, user preference information including hobby information, health condition information, physical activity information, and location preference information from a terminal device, convert the received information into electronic data, and store the electronic data in a storage device;
obtain history information from the terminal device or from an external data source via the communication interface, analyze the stored user preference information and the history information to predict a behavioral pattern of the user, integrate an analysis result to generate a user profile, and store the user profile in the storage device;
acquire contextual data via the communication interface, generate a prompt sentence incorporating the user profile and the contextual data for input to a generative AI model, and obtain from the generative AI model an optimal activity plan for the user; and
transmit the optimal activity plan to the terminal device via the communication interface.

2. The system according to claim 1, wherein the circuitry is configured to receive hobby information, health condition information, preference information, physical activity information, and location preference information from the terminal device, and convert the received information into structured electronic data for storage in the storage device.

3. The system according to claim 2, wherein the circuitry is configured to obtain history information representing past life contents of the user from the terminal device or from an external data source via the communication interface, and incorporate the history information into the user profile.

4. The system according to claim 3, wherein the circuitry is configured to obtain additional information from an external information source via the communication interface, integrate the additional information with the user preference information and the history information, and analyze the integrated data to predict a behavioral pattern of the user.

5. The system according to claim 4, wherein the circuitry is configured to acquire weather information via the communication interface, incorporate the weather information into the prompt sentence for the generative AI model, and generate an optimal activity plan that accounts for current weather conditions.

6. The system according to claim 5, wherein the circuitry is configured to generate the prompt sentence incorporating at least the user profile, the history information, the additional information, and the weather information, and obtain from the generative AI model an activity plan optimized across the integrated data.

7. The system according to claim 1, wherein the circuitry is configured to apply a machine learning algorithm to the stored user preference information and history information to identify patterns in the behavioral data, and use the identified patterns to generate the user profile.

8. The system according to claim 7, wherein the circuitry is configured to update the user profile stored in the storage device based on newly received user preference information and history information, and regenerate the prompt sentence for the generative AI model based on the updated user profile.

9. The system according to claim 1, wherein the circuitry is configured to receive feedback from the terminal device indicating whether the transmitted activity plan was accepted or modified by the user, and incorporate the feedback into the user profile stored in the storage device.

10. The system according to claim 9, wherein the circuitry is configured to update the behavioral pattern prediction based on the feedback, and adjust the prompt sentence for subsequent activity plan generation to reflect the updated prediction.

11. The system according to claim 1, wherein the circuitry is configured to receive health metric data from the terminal device via the communication interface, integrate the health metric data with the user profile, and incorporate the integrated data into the prompt sentence for generating a health-aware activity plan.

12. The system according to claim 11, wherein the circuitry is configured to detect changes in the health metric data over time, update the health condition information in the user profile stored in the storage device, and generate an updated prompt sentence for the generative AI model based on the updated health condition information.

13. The system according to claim 1, wherein the circuitry is configured to acquire service usage records from an external data source via the communication interface, and incorporate the service usage records into the integrated data used for behavioral pattern prediction and user profile generation.

14. The system according to claim 13, wherein the circuitry is configured to analyze temporal patterns in the service usage records, identify recurring preferences or behaviors, and incorporate the identified patterns into the prompt sentence for generating a personalized activity plan.

15. The system according to claim 1, wherein the circuitry is configured to generate a plurality of candidate activity plans by generating a plurality of prompt sentences with different parameter settings, evaluate the candidate plans based on consistency with the user profile, and transmit a ranked set of candidate plans to the terminal device.

16. The system according to claim 15, wherein the circuitry is configured to receive a selection of one of the candidate activity plans from the terminal device, store the selected plan in the storage device, and use the selected plan to update the behavioral pattern prediction.

17. The system according to claim 1, wherein the circuitry is configured to monitor changes in the contextual data received from the terminal device over time, detect significant changes in the contextual data, and regenerate the prompt sentence for the generative AI model when a detected change exceeds a threshold.

18. A system comprising:
circuitry configured to:
receive, via a communication interface coupled to a packet-switched network, user preference information including hobby information, health condition information, physical activity information, and location preference information from a terminal device, and store the user preference information in a storage device;
obtain history information and additional information from external data sources via the communication interface, analyze the integrated data to predict a behavioral pattern of the user, and generate a user profile stored in the storage device;
acquire contextual data via the communication interface, generate a prompt sentence incorporating the user profile and the contextual data, input the prompt sentence to a generative AI model, and obtain an optimal activity plan; and
transmit the optimal activity plan to the terminal device via the communication interface, and update the user profile based on feedback received from the terminal device.

19. The system according to claim 18, wherein the circuitry is configured to acquire weather information via the communication interface, incorporate the weather information into the prompt sentence for the generative AI model, and generate an activity plan that accounts for current weather conditions in combination with the user profile.

20. A method comprising:
receiving, via a communication interface coupled to a packet-switched network, user preference information from a terminal device, converting the received information into electronic data, and storing the electronic data in a storage device;
obtaining history information from the terminal device or from an external data source via the communication interface, analyzing the stored user preference information and the history information to predict a behavioral pattern of the user, and generating a user profile;
acquiring contextual data via the communication interface, generating a prompt sentence incorporating the user profile and the contextual data for input to a generative AI model, and obtaining an optimal activity plan from the generative AI model; and
transmitting the optimal activity plan to the terminal device via the communication interface.
